(19) United States
(12) Patent Application Publication
(10) Pub. No.: US 2011/0217706 A1
(43) Pub. Date: Sep. 8, 2011

(54) GENE METHYLATION IN CANCER DIAGNOSIS

(76) Inventors: Rebecca Maloney, St. Louis, MO (US); Arief Budiman, Manchester, MO (US); Yulia Korshunova, Clayton, MO (US); Jared Ordway, St. Louis, MO (US)

(21) Appl. No.: 13/058,191

(22) PCT Filed: Aug. 7, 2009
(86) PCT No.: PCT/US2009/053122

§ 371 Date: May 20, 2011

Related U.S. Application Data
(60) Provisional application No. 61/087,530, filed on Aug. 8, 2008.

(51) Int. Cl. C12Q 1/68; G06F 19/00

(52) U.S. Cl. 435/6.11; 702/19

(57) ABSTRACT

DNA biomarker sequences that are differentially methylated in samples from normal individuals and individuals with cancer are provided Additionally, methods of identifying differentially methylated DNA biomarker sequences and their use for the detection and diagnosis of cancer are provided.

### CROSS-REFERENCE TO RELATED PATENT APPLICATIONS

The present application claims benefit of priority to U.S. Provisional Patent Application No. 61/087,530, filed Aug. 8, 2008, which is incorporated by reference for all purposes.

### BACKGROUND OF THE INVENTION

Human cancer cells typically contain somatically altered genomes, characterized by mutation, amplification, or deletion of critical genes. In addition, the DNA template from human cancer cells often displays somatic changes in DNA methylation. See, e.g., E. R. Fearon, et al, Cell 61:759 (1990); P. A. Jones, et al., Cancer Res. 46:461 (1986); R. Holliday, Science 238:163 (1987); A. De Bustros, et al., Proc. Natl. Acad. Sci. USA 85:5693 (1988); P. A. Jones, et al., Adv. Cancer Res. 54:1 (1990); S. B. Baylin, et al., Cancer Cells 3:383 (1991); M. Makos, et al., Proc. Natl. Acad Sci. USA 89:1929 (1992); N. Ohtani-Fujita, et al., Oncogene 8:1063 (1993).

DNA methylases transfer methyl groups from the universal methyl donor S-adenosyl methionine to specific sites on the DNA. Several biological functions have been attributed to the methylated bases in DNA. The most established biological function is the protection of the DNA from digestion by cognate restriction enzymes. This restriction modification phenomenon has, so far, been observed only in bacteria.

Mammalian cells, however, possess different methylases that exclusively methylate cytosine residues on the DNA that are 5′ neighbors of guanine (CpG). This methylation has been shown by several lines of evidence to play a role in gene activity, cell differentiation, tumorigenesis, X-chromosome inactivation, genomic imprinting and other major biological processes (Razin, A., H., and Riggs, R. D. eds. in DNA Methylation Biochemistry and Biological Significance, Springer-Verlag, N.Y., 1984).

In eukaryotic cells, methylation of cytosine residues that are immediately 5′ to a guanosine, occurs predominantly in CG poor loci (Bird, A., Nature 321:209 (1986)). In contrast, discrete regions of CG dinucleotides called CG islands (CGi) remain unmethylated in normal cells, except during X-chromosome inactivation and parental specific imprinting (Li, et al., Nature 366:362 (1993)) where methylation of 5′ regulatory regions can lead to transcriptional repression. For example, de novo methylation of the Rb gene has been demonstrated in a small fraction of retinoblastomas (Sakai, et al., Am. J. Hum. Genet., 48:880 (1991)), and a more detailed analysis of the VHL gene showed aberrant methylation in a subset of sporadic renal cell carcinomas (Herman, et al., Proc. Natl. Acad. Sci. U.S.A., 91:9700 (1994)). Expression of a tumor suppressor gene can also be abolished by de novo DNA methylation of a normally unmethylated 5′ CG island. See, e.g., Issa, et al., Nature Genet. 7:536 (1994); Merlo, et al., Nature Med. 1:686 (1995); Herman, et al., Cancer Res., 56:722 (1996); Graff, et al., Cancer Res., 55:5195 (1995); Herman, et al., Cancer Res. 55:4525 (1995).

Identification of the earliest genetic and epigenetic changes in tumorigenesis is a major focus in molecular cancer research. Diagnostic approaches based on identification of these changes can allow implementation of early detection strategies, tumor staging, and novel therapeutic approaches targeting these early changes, all of which lead to more effective cancer treatment. The present invention addresses these and other problems.

### BRIEF SUMMARY OF THE INVENTION

The present invention provides methods for determining the methylation status of an individual. In one aspect, the methods comprise:

obtaining a biological sample from an individual; and

determining the methylation status of at least one cytosine within a DNA region in a sample from an individual where the DNA region is at least 90%, 91%, 92%, 93%, 94%, 95%, 96%, 97%, 98%, or 99% identical to, or comprises, a sequence selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480.

In a further aspect, the methods comprise determining (e.g. correlating methylation status to) the presence or absence of cancer, including but not limited to, bladder, breast, cervical, colon, endometrial, esophageal, head and neck, liver, lung, melanoma, ovarian, prostate, renal, and thyroid cancer, in an individual.

In some embodiments, the methods comprise:

a) determining the methylation status of at least one cytosine within a DNA region in a sample from the individual where the DNA region is at least 90%, 91%, 92%, 93%, 94%, 95%, 96%, 97%, 98%, or 99% identical to, or comprises, a sequence selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480;

b) comparing the methylation status of the at least one cytosine to a threshold value for the biomarker, wherein the threshold value distinguishes between individuals with and without cancer, wherein the comparison of the methylation status to the threshold value is predictive of the presence or absence of cancer in the individual.

In some embodiments, the methods comprise:

a) determining the methylation status of at least one cytosine within a DNA region in a sample from the individual where the DNA region is at least 90%, 91%, 92%, 93%, 94%, 95%, 96%, 97%, 98%, or 99% identical to, or comprises, a sequence selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480;

b) comparing the methylation status of the at least one cytosine to a threshold value for the biomarker, wherein the threshold value distinguishes between individuals with and without bladder cancer, wherein the comparison of the methylation status to the threshold value is predictive of the presence or absence of bladder cancer in the individual.

In some embodiments, the methods comprise:

a) determining the methylation status of at least one cytosine within a DNA region in a sample from the individual where the DNA region is at least 90%, 91%, 92%, 93%, 94%, 95%, 96%, 97%, 98%, or 99% identical to, or comprises, a sequence selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480;

b) comparing the methylation status of the at least one cytosine to a threshold value for the biomarker, wherein the threshold value distinguishes between individuals with and without breast cancer, wherein the comparison of the methylation status to the threshold value is predictive of the presence or absence of breast cancer in the individual.

In some embodiments, the methods comprise:

a) determining the methylation status of at least one cytosine within a DNA region in a sample from the individual where the DNA region is at least 90%, 91%, 92%, 93%, 94%, 95%, 96%, 97%, 98%, or 99% identical to, or comprises, a sequence selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480;

b) comparing the methylation status of the at least one cytosine to a threshold value for the biomarker, wherein the threshold value distinguishes between individuals with and without cervical cancer, wherein the comparison of the methylation status to the threshold value is predictive of the presence or absence of cervical cancer in the individual.

In some embodiments, the methods comprise:

a) determining the methylation status of at least one cytosine within a DNA region in a sample from the individual where the DNA region is at least 90%, 91%, 92%, 93%, 94%, 95%, 96%, 97%, 98%, or 99% identical to, or comprises, a sequence selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480;

b) comparing the methylation status of the at least one cytosine to a threshold value for the biomarker, wherein the threshold value distinguishes between individuals with and without colon cancer, wherein the comparison of the methylation status to the threshold value is predictive of the presence or absence of colon cancer in the individual.

In some embodiments, the methods comprise:

a) determining the methylation status of at least one cytosine within a DNA region in a sample from the individual where the DNA region is at least 90%, 91%, 92%, 93%, 94%, 95%, 96%, 97%, 98%, or 99% identical to, or comprises, a sequence selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480;

b) comparing the methylation status of the at least one cytosine to a threshold value for the biomarker, wherein the threshold value distinguishes between individuals with and without endometrial cancer, wherein the comparison of the methylation status to the threshold value is predictive of the presence or absence of endometrial cancer in the individual.

In some embodiments, the methods comprise:

a) determining the methylation status of at least one cytosine within a DNA region in a sample from the individual where the DNA region is at least 90%, 91%, 92%, 93%, 94%, 95%, 96%, 97%, 98%, or 99% identical to, or comprises, a sequence selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480;

b) comparing the methylation status of the at least one cytosine to a threshold value for the biomarker, wherein the threshold value distinguishes between individuals with and without esophageal cancer, wherein the comparison of the methylation status to the threshold value is predictive of the presence or absence of esophageal cancer in the individual.

In some embodiments, the methods comprise:

a) determining the methylation status of at least one cytosine within a DNA region in a sample from the individual where the DNA region is at least 90%, 91%, 92%, 93%, 94%, 95%, 96%, 97%, 98%, or 99% identical to, or comprises, a sequence selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480;

b) comparing the methylation status of the at least one cytosine to a threshold value for the biomarker, wherein the threshold value distinguishes between individuals with and without head and neck cancer, wherein the comparison of the methylation status to the threshold value is predictive of the presence or absence of head and neck cancer in the individual.

In some embodiments, the methods comprise:

a) determining the methylation status of at least one cytosine within a DNA region in a sample from the individual where the DNA region is at least 90%, 91%, 92%, 93%, 94%, 95%, 96%, 97%, 98%, or 99% identical to, or comprises, a sequence selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480;

b) comparing the methylation status of the at least one cytosine to a threshold value for the biomarker, wherein the threshold value distinguishes between individuals with and without liver cancer, wherein the comparison of the methylation status to the threshold value is predictive of the presence or absence of liver cancer in the individual.

In some embodiments, the methods comprise:

a) determining the methylation status of at least one cytosine within a DNA region in a sample from the individual where the DNA region is at least 90%, 91%, 92%, 93%, 94%, 95%, 96%, 97%, 98%, or 99% identical to, or comprises, a sequence selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480;

b) comparing the methylation status of the at least one cytosine to a threshold value for the biomarker, wherein the threshold value distinguishes between individuals with and without lung cancer, wherein the comparison of the methylation status to the threshold value is predictive of the presence or absence of lung cancer in the individual.

In some embodiments, the methods comprise:

a) determining the methylation status of at least one cytosine within a DNA region in a sample from the individual where the DNA region is at least 90%, 91%, 92%, 93%, 94%, 95%, 96%, 97%, 98%, or 99% identical to, or comprises, a sequence selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480;

b) comparing the methylation status of the at least one cytosine to a threshold value for the biomarker, wherein the threshold value distinguishes between individuals with and without melanoma, wherein the comparison of the methylation status to the threshold value is predictive of the presence or absence of melanoma in the individual.

In some embodiments, the methods comprise:

a) determining the methylation status of at least one cytosine within a DNA region in a sample from the individual where the DNA region is at least 90%, 91%, 92%, 93%, 94%, 95%, 96%, 97%, 98%, or 99% identical to, or comprises, a sequence selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480;

b) comparing the methylation status of the at least one cytosine to a threshold value for the biomarker, wherein the threshold value distinguishes between individuals with and without ovarian cancer, wherein the comparison of the methylation status to the threshold value is predictive of the presence or absence of ovarian cancer in the individual.

In some embodiments, the methods comprise:

a) determining the methylation status of at least one cytosine within a DNA region in a sample from the individual where the DNA region is at least 90%, 91%, 92%, 93%, 94%, 95%, 96%, 97%, 98%, or 99% identical to, or comprises, a sequence selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480;

b) comparing the methylation status of the at least one cytosine to a threshold value for the biomarker, wherein the threshold value distinguishes between individuals with and without prostate cancer, wherein the comparison of the methylation status to the threshold value is predictive of the presence or absence of prostate cancer in the individual.

In some embodiments, the methods comprise:

a) determining the methylation status of at least one cytosine within a DNA region in a sample from the individual where the DNA region is at least 90%, 91%, 92%, 93%, 94%, 95%, 96%, 97%, 98%, or 99% identical to, or comprises, a sequence selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480;

b) comparing the methylation status of the at least one cytosine to a threshold value for the biomarker, wherein the threshold value distinguishes between individuals with and without renal cancer, wherein the comparison of the methylation status to the threshold value is predictive of the presence or absence of renal cancer in the individual.

In some embodiments, the methods comprise:

a) determining the methylation status of at least one cytosine within a DNA region in a sample from the individual where the DNA region is at least 90%, 91%, 92%, 93%, 94%, 95%, 96%, 97%, 98%, or 99% identical to, or comprises, a sequence selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480;

b) comparing the methylation status of the at least one cytosine to a threshold value for the biomarker, wherein the threshold value distinguishes between individuals with and without thyroid cancer, wherein the comparison of the methylation status to the threshold value is predictive of the presence or absence of thyroid cancer in the individual.

With regard to the embodiments, in some embodiments, the determining step comprises determining the methylation status of at least one cytosine in the DNA region corresponding to a nucleotide in a biomarker, wherein the biomarker is at least 90%, 91%, 92%, 93%, 94%, 95%, 96%, 97%, 98%, or 99% identical to, or comprises, a sequence selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480.

In some embodiments, the determining step comprises determining the methylation status of the DNA region corresponding to a biomarker.

The sample can be from any body fluid. In some embodiments, the sample is selected from blood serum, blood plasma, fine needle aspirate of the breast, biopsy of the breast, ductal fluid, ductal lavage, feces, urine, sputum, saliva, semen, lavages, or tissue biopsy, such as biopsy of the lung, bronchial lavage or bronchial brushings in the case of lung cancer. In some embodiments, the sample is from a tumor or polyp. In some embodiments, the sample is a biopsy from lung, kidney, liver, ovarian, head, neck, thyroid, bladder, cervical, colon, endometrial, esophageal, prostate or skin tissue. In some embodiments, the sample is from cell scrapes, washings, or resected tissues.

In some embodiments, the methylation status of at least one cytosine is compared to the methylation status of a control locus. In some embodiments, the control locus is an endogenous control. In some embodiments, the control locus is an exogenous control.

In some embodiments, the determining step comprises determining the methylation status of at least one cytosine in at least two of the DNA regions.

In a further aspect, the invention provides computer implemented methods for determining the presence or absence of cancer (including but not limited to cancers of the bladder, breast, cervix, colon, endometrium, esophagus, head and neck, liver, lung(s), ovaries, prostate, rectum, and thyroid, and melanoma) in an individual. In some embodiments, the methods comprise:

receiving, at a host computer, a methylation value representing the methylation status of at least one cytosine within a DNA region in a sample from the individual where the DNA region is at least 90%, 91%, 92%, 93%, 94%, 95%, 96%, 97%, 98%, or 99% identical to, or comprises, a sequence is selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480; and

comparing, in the host computer, the methylation value to a threshold value, wherein the threshold value distinguishes between individuals with and without cancer (including but not limited to cancers of the bladder, breast, cervix, colon, endometrium, esophagus, head and neck, liver, lung(s), ovaries, prostate, rectum, and thyroid, and melanoma), wherein the comparison of the methylation value to the threshold value is predictive of the presence or absence of cancer (including but not limited to cancers of the bladder, breast, cervix, colon, endometrium, esophagus, head and neck, liver, lung(s), ovaries, prostate, rectum, and thyroid, and melanoma) in the individual.

In some embodiments, the receiving step comprises receiving at least two methylation values, the two methylation values representing the methylation status of at least one cytosine biomarkers from two different DNA regions; and

the comparing step comprises comparing the methylation values to one or more threshold value(s) wherein the threshold value distinguishes between individuals with and without cancer (including but not limited to cancers of the bladder, breast, cervix, colon, endometrium, esophagus, head and neck, liver, lung(s), ovaries, prostate, rectum, and thyroid, and melanoma), wherein the comparison of the methylation value to the threshold value is predictive of the presence or absence of cancer (including but not limited to cancers of the bladder, breast, cervix, colon, endometrium, esophagus, head and neck, liver, lung(s), ovaries, prostate, rectum, and thyroid, and melanoma) in the individual.

In another aspect, the invention provides computer program products for determining the presence or absence of cancer (including but not limited to cancers of the bladder, breast, cervix, colon, endometrium, esophagus, head and neck, liver, lung(s), ovaries, prostate, rectum, and thyroid, and melanoma) in an individual. In some embodiments, the computer readable products comprise:

a computer readable medium encoded with program code, the program code including:

program code for receiving a methylation value representing the methylation status of at least one cytosine within a DNA region in a sample from the individual where the DNA region is at least 90%, 91%, 92%, 93%, 94%, 95%, 96%, 97%, 98%, or 99% identical to, or comprises, a sequence selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480; and

program code for comparing the methylation value to a threshold value, wherein the threshold value distinguishes between individuals with and without cancer (including but not limited to cancers of the bladder, breast, cervix, colon, endometrium, esophagus, head and neck, liver, lung(s), ovaries, prostate, rectum, and thyroid, and melanoma), wherein the comparison of the methylation value to the threshold value is predictive of the presence or absence of cancer (including but not limited to cancers of the bladder, breast, cervix, colon, endometrium, esophagus, head and neck, liver, lung(s), ovaries, prostate, rectum, and thyroid, and melanoma) in the individual.

In a further aspect, the invention provides kits for determining the methylation status of at least one biomarker. In some embodiments, the kits comprise:

a pair of polynucleotides capable of specifically amplifying at least a portion of a DNA region where the DNA region is selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480; and

a methylation-dependent or methylation sensitive restriction enzyme and/or sodium bisulfite.

In some embodiments, the pair of polynucleotides are capable of specifically amplifying a biomarker selected from the group consisting of one or more of SEQ ID NOS:289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383 and 384.

In some embodiments, the kits comprise at least two pairs of polynucleotides, wherein each pair is capable of specifically amplifying at least a portion of a different DNA region.

In some embodiments, the kits further comprise a detectably labeled polynucleotide probe that specifically detects the amplified biomarker in a real time amplification reaction.

In a further aspect, the invention provides kits for determining the methylation status of at least one biomarker. In some embodiments, the kits comprise:

sodium bisulfite and polynucleotides to quantify the presence of the converted methylated and/or the converted unmethylated sequence of at least one cytosine from a DNA region that is selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480.

In a further aspect, the invention provides kits for determining the methylation status of at least one biomarker. In some embodiments, the kits comprise:

sodium bisulfite, primers and adapters for whole genome amplification, and polynucleotides to quantify the presence of the converted methylated and/or the converted unmethylated sequence of at least one cytosine from a DNA region that is selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480.

In another aspect, the methods provide kits for determining the methylation status of at least one biomarker. In some embodiments, the kits comprise:

a methylation sensing restriction enzymes, primers and adapters for whole genome amplification, and polynucleotides to quantify the number of copies of at least a portion of a DNA region where the DNA region is at least 90%, 91%, 92%, 93%, 94%, 95%, 96%, 97%, 98%, or 99% identical to, or comprises, a sequence selected from the group consisting of SEQ ID NO: 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480.

In a further aspect, the invention provides kits for determining the methylation status of at least one biomarker. In some embodiments, the kits comprise:

a methylation sensing binding moiety and polynucleotides to quantify the number of copies of at least a portion of a DNA region where the DNA region is at least 90%, 91%, 92%, 93%, 94%, 95%, 96%, 97%, 98%, or 99% identical to, or comprises, a sequence selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480.

### DEFINITIONS

“Methylation” refers to cytosine methylation at positions C5 or N4 of cytosine, the N6 position of adenine or other types of nucleic acid methylation. In vitro amplified DNA is unmethylated because in vitro DNA amplification methods do not retain the methylation pattern of the amplification template. However, “unmethylated DNA” or “methylated DNA” can also refer to amplified DNA whose original template was unmethylated or methylated, respectively.

A “methylation profile” refers to a set of data representing the methylation states of one or more loci within a molecule of DNA from e.g., the genome of an individual or cells or tissues from an individual. The profile can indicate the methylation state of every base in an individual, can comprise information regarding a subset of the base pairs (e.g., the methylation state of specific restriction enzyme recognition sequence) in a genome, or can comprise information regarding regional methylation density of each locus.

“Methylation status” refers to the presence, absence and/or quantity of methylation at a particular nucleotide, or nucleotides within a portion of DNA. The methylation status of a particular DNA sequence (e.g., a DNA biomarker or DNA region as described herein) can indicate the methylation state of every base in the sequence or can indicate the methylation state of a subset of the base pairs (e.g., of cytosines or the methylation state of one or more specific restriction enzyme recognition sequences) within the sequence, or can indicate information regarding regional methylation density within the sequence without providing precise information of where in the sequence the methylation occurs. The methylation status can optionally be represented or indicated by a “methylation value.” A methylation value can be generated, for example, by quantifying the amount of intact DNA present following restriction digestion with a methylation dependent restriction enzyme. In this example, if a particular sequence in the DNA is quantified using quantitative PCR, an amount of template DNA approximately equal to a mock treated control indicates the sequence is not highly methylated whereas an amount of template substantially less than occurs in the mock treated sample indicates the presence of methylated DNA at the sequence. Accordingly, a value, i.e., a methylation value from the above described example represents the methylation status and can thus be used as a quantitative indicator of methylation status. This is of particular use when it is desirable to compare the methylation status of a sequence in a sample to a threshold value.

A “methylation-dependent restriction enzyme” refers to a restriction enzyme that cleaves or digests DNA at or in proximity to a methylated recognition sequence, but does not cleave DNA at or near the same sequence when the recognition sequence is not methylated. Methylation-dependent restriction enzymes include those that cut at a methylated recognition sequence (e.g., DpnI) and enzymes that cut at a sequence near but not at the recognition sequence (e.g., McrBC). For example, McrBC's recognition sequence is 5′ RmC (N40-3000) RmC 3′ where “R” is a purine and “mC” is a methylated cytosine and “N40-3000” indicates the distance between the two RmC half sites for which a restriction event has been observed. McrBC generally cuts close to one half-site or the other, but cleavage positions are typically distributed over several base pairs, approximately 30 base pairs from the methylated base. McrBC sometimes cuts 3′ of both half sites, sometimes 5′ of both half sites, and sometimes between the two sites. Exemplary methylation-dependent restriction enzymes include, e.g., McrBC (see, e.g., U.S. Pat. No. 5,405,760), McrA, MrrA, BisI, GlaI and DpnI. One of skill in the art will appreciate that any methylation-dependent restriction enzyme, including homologs and orthologs of the restriction enzymes described herein, is also suitable for use in the present invention.

A “methylation-sensitive restriction enzyme” refers to a restriction enzyme that cleaves DNA at or in proximity to an unmethylated recognition sequence but does not cleave at or in proximity to the same sequence when the recognition sequence is methylated. Exemplary methylation-sensitive restriction enzymes are described in, e.g., McClelland et al., Nucleic Acids Res. 22 (17):3640-59 (1994) and http://rebase.neb.com. Suitable methylation-sensitive restriction enzymes that do not cleave DNA at or near their recognition sequence when a cytosine within the recognition sequence is methylated at position C⁵ include, e.g., Aat II, Aci I, Acl I, Age I, Alu I, Asc I, Ase I, AsiS I, Bbe I, BsaA I, BsaH I, BsiE I, BsiW I, BsrF I, BssH II, BssK I, BstB I, BstN I, BstU I, Cla I, Eae I, Eag I, Fau I, Fse I, Hha I, HinP1 I, HinC II, Hpa II, Hpy99 I, HpyCH4 IV, Kas I, Mbo I, Mlu I, MapA1 I, Msp I, Nae I, Nar I, Not I, Pml I, Pst I, Pvu I, Rsr II, Sac II, Sap I, Sau3A I, Sfl I, Sfo I, SgrA I, Sma I, SnaB I, Tsc I, Xma I, and Zra I. Suitable methylation-sensitive restriction enzymes that do not cleave DNA at or near their recognition sequence when an adenosine within the recognition sequence is methylated at position N⁶ include, e.g., Mbo I. One of skill in the art will appreciate that any methylation-sensitive restriction enzyme, including homologs and orthologs of the restriction enzymes described herein, is also suitable for use in the present invention. One of skill in the art will further appreciate that a methylation-sensitive restriction enzyme that fails to cut in the presence of methylation of a cytosine at or near its recognition sequence may be insensitive to the presence of methylation of an adenosine at or near its recognition sequence. Likewise, a methylation-sensitive restriction enzyme that fails to cut in the presence of methylation of an adenosine at or near its recognition sequence may be insensitive to the presence of methylation of a cytosine at or near its recognition sequence. For example, Sau3AI is sensitive (i.e., fails to cut) to the presence of a methylated cytosine at or near its recognition sequence, but is insensitive (i.e., cuts) to the presence of a methylated adenosine at or near its recognition sequence. One of skill in the art will also appreciate that some methylation-sensitive restriction enzymes are blocked by methylation of bases on one or both strands of DNA encompassing of their recognition sequence, while other methylation-sensitive restriction enzymes are blocked only by methylation on both strands, but can cut if a recognition site is hemi-methylated.

A “threshold value that distinguishes between individuals with and without” a particular disease refers to a value or range of values of a particular measurement that can be used to distinguish between samples from individuals with the disease and samples without the disease. Ideally, there is a threshold value or values that absolutely distinguishes between the two groups (i.e., values from the diseased group are always on one side (e.g., higher) of the threshold value and values from the healthy, non-diseased group are on the other side (e.g., lower) of the threshold value). However, in many instances, threshold values do not absolutely distinguish between diseased and non-diseased samples (for example, when there is some overlap of values generated from diseased and non-diseased samples).

The phrase “corresponding to a nucleotide in a biomarker” refers to a nucleotide in a DNA region that aligns with the same nucleotide (e.g., a cytosine) in a biomarker sequence. Generally, as described herein, biomarker sequences are subsequences of (i.e., have 100% identity with) the DNA regions. Sequence comparisons can be performed using any BLAST including BLAST 2.2 algorithm with default parameters, described in Altschul et al., Nuc. Acids Res. 25:3389 3402 (1977) and Altschul et al., J. Mol. Biol. 215:403 410 (1990), respectively.

“Sensitivity” of a given biomarker refers to the percentage of tumor samples that report a DNA methylation value above a threshold value that distinguishes between tumor and non-tumor samples. The percentage is calculated as follows:

$$Sensitivity = [ \frac{( the number of tumor samples above the threshold )}{( the total number of tumor samples tested )} ] \times 100$$

The equation may also be stated as follows:

$$Sensitivity = [ \frac{( the number of true positive samples )}{\begin{matrix}( the number of true positive samples ) + \\ ( the number of false negative samples )\end{matrix}} ] \times 100$$

where true positive is defined as a histology-confirmed tumor sample that reports a DNA methylation value above the threshold value (i.e. the range associated with disease), and false negative is defined as a histology-confirmed tumor sample that reports a DNA methylation value below the threshold value (i.e. the range associated with no disease). The value of sensitivity, therefore, reflects the probability that a DNA methylation measurement for a given biomarker obtained from a known diseased sample will be in the range of disease-associated measurements. As defined here, the clinical relevance of the calculated sensitivity value represents an estimation of the probability that a given biomarker would detect the presence of a clinical condition when applied to a patient with that condition.

“Specificity” of a given biomarker refers to the percentage of non-tumor samples that report a DNA methylation value below a threshold value that distinguishes between tumor and non-tumor samples. The percentage is calculated as follows:

$$Specificity = [ \frac{( the number of non \text{-} tumor samples below the threshold )}{( the total number of non \text{-} tumor samples tested )} ] \times 100$$

The equation may also be stated as follows:

$$Specificity = [ \frac{( the number of true negative samples )}{\begin{matrix}( the number of true negative samples ) + \\ ( the number of false positive samples )\end{matrix}} ] \times 100$$

where true negative is defined as a histology-confirmed non-tumor sample that reports a DNA methylation value below the threshold value (i.e. the range associated with no disease), and false positive is defined as a histology-confirmed non-tumor sample that reports DNA methylation value above the threshold value (i.e. the range associated with disease). The value of specificity, therefore, reflects the probability that a DNA methylation measurement for a given biomarker obtained from a known non-diseased sample will be in the range of non-disease associated measurements. As defined here, the clinical relevance of the calculated specificity value represents an estimation of the probability that a given biomarker would detect the absence of a clinical condition when applied to a patient without that condition.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

As used herein, the terms “nucleic acid,” “polynucleotide” and “oligonucleotide” refer to nucleic acid regions, nucleic acid segments, primers, probes, amplicons and oligomer fragments. The terms are not limited by length and are generic to linear polymers of polydeoxyribonucleotides (containing 2-deoxy-D-ribose), polyribonucleotides (containing D-ribose), and any other N-glycoside of a purine or pyrimidine base, or modified purine or pyrimidine bases. These terms include double- and single-stranded DNA, as well as double- and single-stranded RNA.

A nucleic acid, polynucleotide or oligonucleotide can comprise, for example, phosphodiester linkages or modified linkages including, but not limited to phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages.

A nucleic acid, polynucleotide or oligonucleotide can comprise the five biologically occurring bases (adenine, guanine, thymine, cytosine and uracil) and/or bases other than the five biologically occurring bases. For example, a polynucleotide of the invention can contain one or more modified, non-standard, or derivatized base moieties, including, but not limited to, N⁶-methyl-adenine, N⁶-tert-butyl-benzyl-adenine, imidazole, substituted imidazoles, 5-fluorouracil, 5-bromouracil, 5-chlorouracil, 5-iodouracil, hypoxanthine, xanthine, 4-acetylcytosine, 5-(carboxyhydroxymethyl)uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-methyladenine, 7-methylguanine, 5-methylaminomethyluracil, 5-methoxyaminomethyl-2-thiouracil, beta-D mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, uracil-5-oxyacetic acidmethylester, 3-(3-amino-3-N-2-carboxypropyl) uracil, (acp3)w, 2,6-diaminopurine, and 5-propynyl pyrimidine. Other examples of modified, non-standard, or derivatized base moieties may be found in U.S. Pat. Nos. 6,001,611; 5,955,589; 5,844,106; 5,789,562; 5,750,343; 5,728,525; and 5,679,785.

Furthermore, a nucleic acid, polynucleotide or oligonucleotide can comprise one or more modified sugar moieties including, but not limited to, arabinose, 2-fluoroarabinose, xylulose, and a hexose.

### BRIEF DESCRIPTION OF THE DRAWINGS

### I. Introduction

The present invention is based, in part, on the discovery that sequences in certain DNA regions are methylated in cancer cells, but not normal cells. Specifically, the inventors have found that methylation of biomarkers within the DNA regions described herein are associated with various types of cancer.

In view of this discovery, the inventors have recognized that methods for detecting the biomarker sequences and DNA regions comprising the biomarker sequences as well as sequences adjacent to the biomarkers that contain a significant amount of CG subsequences, methylation of the DNA regions, and/or expression of the genes regulated by the DNA regions can be used to detect cancer cells. Detecting cancer cells allows for diagnostic tests that detect disease, assess the risk of contracting disease, determining a predisposition to disease, stage disease, diagnose disease, monitor disease, and/or aid in the selection of treatment for a person with disease.

### II. Methylation Biomarkers

In some embodiments, the presence or absence or quantity of methylation of the chromosomal DNA within a DNA region or portion thereof (e.g., at least one cytosine) selected from SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480 is detected. Portions of the DNA regions described herein will comprise at least one potential methylation site (i.e., a cytosine) and can in some embodiments generally comprise 2, 3, 4, 5, 10, or more potential methylation sites. In some embodiments, the methylation status of all cytosines within at least 20, 50, 100, 200, 500 or more contiguous base pairs of the DNA region are determined.

In some embodiments, the methylation of more than one DNA region (or portion thereof) is detected. In some embodiments, the methylation status of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96 of the DNA regions is determined.

In some embodiments of the invention, the methylation of a DNA region or portion thereof is determined and then normalized (e.g., compared) to the methylation of a control locus. Typically the control locus will have a known, relatively constant, methylation status. For example, the control sequence can be previously determined to have no, some or a high amount of methylation, thereby providing a relative constant value to control for error in detection methods, etc., unrelated to the presence or absence of cancer. In some embodiments, the control locus is endogenous, i.e., is part of the genome of the individual sampled. For example, in mammalian cells, the testes-specific histone 2B gene (hTH2B in human) gene is known to be methylated in all somatic tissues except testes. Alternatively, the control locus can be an exogenous locus, i.e., a DNA sequence spiked into the sample in a known quantity and having a known methylation status.

A DNA region comprises a nucleic acid including one or more methylation sites of interest (e.g., a cytosine, a “microarray feature,” or an amplicon amplified from select primers) and flanking nucleic acid sequences (i.e., “wingspan”) of up to 4 kilobases (kb) in either or both of the 3′ or 5′ direction from the amplicon. This range corresponds to the lengths of DNA fragments obtained by randomly fragmenting the DNA before screening for differential methylation between DNA in two or more samples (e.g., carrying out methods used to initially identify differentially methylated sequences as described in the Examples, below). In some embodiments, the wingspan of the one or more DNA regions is about 0.5 kb, 0.75 kb, 1.0 kb, 1.5 kb, 2.0 kb, 2.5 kb, 3.0 kb, 3.5 kb or 4.0 kb in both 3′ and 5′ directions relative to the sequence represented by the microarray feature.

The methylation sites in a DNA region can reside in non-coding transcriptional control sequences (e.g., promoters, enhancers, etc.) or in coding sequences, including introns and exons of the designated genes listed in Tables 1-4 and in the section, “INFORMAL SEQUENCE LISTING.” In some embodiments, the methods comprise detecting the methylation status in the promoter regions (e.g., comprising the nucleic acid sequence that is about 1.0 kb, 1.5 kb, 2.0 kb, 2.5 kb, 3.0 kb, 3.5 kb or 4.0 kb 5′ from the transcriptional start site through to the transcriptional start site) of one or more of the genes identified in Tables 1-4 and in the section, “INFORMAL SEQUENCE LISTING.”

The DNA regions of the invention also include naturally occurring variants, including for example, variants occurring in different subject populations and variants arising from single nucleotide polymorphisms (SNPs). SNPs encompass insertions and deletions of varying size and simple sequence repeats, such as dinucleotides and trinucleotide repeats. Variants include nucleic acid sequences from the same DNA region (e.g., as set forth in Table 4 and in section “INFORMAL SEQUENCE LISTING”) sharing at least 90%, 95%, 98%, 99% sequence identity, i.e., having one or more deletions, additions, substitutions, inverted sequences, etc., relative to the DNA regions described herein.

### III. Methods for Determining Methylation

Any method for detecting DNA methylation can be used in the methods of the present invention.

In some embodiments, methods for detecting methylation include randomly shearing or randomly fragmenting the genomic DNA, cutting the DNA with a methylation-dependent or methylation-sensitive restriction enzyme and subsequently selectively identifying and/or analyzing the cut or uncut DNA. Selective identification can include, for example, separating cut and uncut DNA (e.g., by size) and quantifying a sequence of interest that was cut or, alternatively, that was not cut. See, e.g., U.S. Pat. No. 7,186,512. Alternatively, the method can encompass amplifying intact DNA after restriction enzyme digestion, thereby only amplifying DNA that was not cleaved by the restriction enzyme in the area amplified. See, e.g., U.S. patent application Ser. Nos. 10/971,986; 11/071,013; and 10/971,339. In some embodiments, amplification can be performed using primers that are gene specific. Alternatively, adaptors can be added to the ends of the randomly fragmented DNA, the DNA can be digested with a methylation-dependent or methylation-sensitive restriction enzyme, intact DNA can be amplified using primers that hybridize to the adaptor sequences. In this case, a second step can be performed to determine the presence, absence or quantity of a particular gene in an amplified pool of DNA. In some embodiments, the DNA is amplified using real-time, quantitative PCR.

In some embodiments, the methods comprise quantifying the average methylation density in a target sequence within a population of genomic DNA. In some embodiments, the method comprises contacting genomic DNA with a methylation-dependent restriction enzyme or methylation-sensitive restriction enzyme under conditions that allow for at least some copies of potential restriction enzyme cleavage sites in the locus to remain uncleaved; quantifying intact copies of the locus; and comparing the quantity of amplified product to a control value representing the quantity of methylation of control DNA, thereby quantifying the average methylation density in the locus compared to the methylation density of the control DNA.

The quantity of methylation of a locus of DNA can be determined by providing a sample of genomic DNA comprising the locus, cleaving the DNA with a restriction enzyme that is either methylation-sensitive or methylation-dependent, and then quantifying the amount of intact DNA or quantifying the amount of cut DNA at the DNA locus of interest. The amount of intact or cut DNA will depend on the initial amount of genomic DNA containing the locus, the amount of methylation in the locus, and the number (i.e., the fraction) of nucleotides in the locus that are methylated in the genomic DNA. The amount of methylation in a DNA locus can be determined by comparing the quantity of intact DNA or cut DNA to a control value representing the quantity of intact DNA or cut DNA in a similarly-treated DNA sample. The control value can represent a known or predicted number of methylated nucleotides. Alternatively, the control value can represent the quantity of intact or cut DNA from the same locus in another (e.g., normal, non-diseased) cell or a second locus.

By using at least one methylation-sensitive or methylation-dependent restriction enzyme under conditions that allow for at least some copies of potential restriction enzyme cleavage sites in the locus to remain uncleaved and subsequently quantifying the remaining intact copies and comparing the quantity to a control, average methylation density of a locus can be determined. If the methylation-sensitive restriction enzyme is contacted to copies of a DNA locus under conditions that allow for at least some copies of potential restriction enzyme cleavage sites in the locus to remain uncleaved, then the remaining intact DNA will be directly proportional to the methylation density, and thus may be compared to a control to determine the relative methylation density of the locus in the sample. Similarly, if a methylation-dependent restriction enzyme is contacted to copies of a DNA locus under conditions that allow for at least some copies of potential restriction enzyme cleavage sites in the locus to remain uncleaved, then the remaining intact DNA will be inversely proportional to the methylation density, and thus may be compared to a control to determine the relative methylation density of the locus in the sample. Such assays are disclosed in, e.g., U.S. patent application Ser. No. 10/971,986.

Kits for the above methods can include, e.g., one or more of methylation-dependent restriction enzymes, methylation-sensitive restriction enzymes, amplification (e.g., PCR) reagents, probes and/or primers.

Quantitative amplification methods (e.g., quantitative PCR or quantitative linear amplification) can be used to quantify the amount of intact DNA within a locus flanked by amplification primers following restriction digestion. Methods of quantitative amplification are disclosed in, e.g., U.S. Pat. Nos. 6,180,349; 6,033,854; and 5,972,602, as well as in, e.g., Gibson et al., Genome Research 6:995-1001 (1996); DeGraves, et al., Biotechniques 34 (1):106-10, 112-5 (2003); Deiman B, et al., Mol Biotechnol. 20 (2):163-79 (2002). Amplifications may be monitored in “real time.”

Additional methods for detecting DNA methylation can involve genomic sequencing before and after treatment of the DNA with bisulfite. See, e.g., Frommer et al., Proc. Natl. Acad. Sci. USA 89:1827-1831 (1992). When sodium bisulfite is contacted to DNA, unmethylated cytosine is converted to uracil, while methylated cytosine is not modified.

In some embodiments, restriction enzyme digestion of PCR products amplified from bisulfite-converted DNA is used to detect DNA methylation. See, e.g., Sadri & Hornsby, Nucl. Acids Res. 24:5058-5059 (1996); Xiong & Laird, Nucleic Acids Res. 25:2532-2534 (1997).

In some embodiments, a MethyLight assay is used alone or in combination with other methods to detect DNA methylation (see, Eads et al., Cancer Res. 59:2302-2306 (1999)). Briefly, in the MethyLight process genomic DNA is converted in a sodium bisulfite reaction (the bisulfite process converts unmethylated cytosine residues to uracil). Amplification of a DNA sequence of interest is then performed using PCR primers that hybridize to CpG dinucleotides. By using primers that hybridize only to sequences resulting from bisulfite conversion of unmethylated DNA, (or alternatively to methylated sequences that are not converted) amplification can indicate methylation status of sequences where the primers hybridize. Similarly, the amplification product can be detected with a probe that specifically binds to a sequence resulting from bisulfite treatment of a unmethylated (or methylated) DNA. If desired, both primers and probes can be used to detect methylation status. Thus, kits for use with MethyLight can include sodium bisulfite as well as primers or detectably-labeled probes (including but not limited to Taqman or molecular beacon probes) that distinguish between methylated and unmethylated DNA that have been treated with bisulfite. Other kit components can include, e.g., reagents necessary for amplification of DNA including but not limited to, PCR buffers, deoxynucleotides; and a thermostable polymerase.

In some embodiments, a Ms-SNuPE (Methylation-sensitive Single Nucleotide Primer Extension) reaction is used alone or in combination with other methods to detect DNA methylation (see, Gonzalgo & Jones, Nucleic Acids Res. 25:2529-2531 (1997)). The Ms-SNuPE technique is a quantitative method for assessing methylation differences at specific CpG sites based on bisulfite treatment of DNA, followed by single-nucleotide primer extension (Gonzalgo & Jones, supra). Briefly, genomic DNA is reacted with sodium bisulfite to convert unmethylated cytosine to uracil while leaving 5-methylcytosine unchanged. Amplification of the desired target sequence is then performed using PCR primers specific for bisulfite-converted DNA, and the resulting product is isolated and used as a template for methylation analysis at the CpG site(s) of interest.

Typical reagents (e.g., as might be found in a typical Ms-SNuPE-based kit) for Ms-SNuPE analysis can include, but are not limited to: PCR primers for specific gene (or methylation-altered DNA sequence or CpG island); optimized PCR buffers and deoxynucleotides; gel extraction kit; positive control primers; Ms-SNuPE primers for a specific gene; reaction buffer (for the Ms-SNuPE reaction); and detectably-labeled nucleotides. Additionally, bisulfite conversion reagents may include: DNA denaturation buffer; sulfonation buffer; DNA recovery regents or kit (e.g., precipitation, ultrafiltration, affinity column); desulfonation buffer; and DNA recovery components.

In some embodiments, a methylation-specific PCR (“MSP”) reaction is used alone or in combination with other methods to detect DNA methylation. An MSP assay entails initial modification of DNA by sodium bisulfite, converting all unmethylated, but not methylated, cytosines to uracil, and subsequent amplification with primers specific for methylated versus unmethylated DNA. See, Herman et al., Proc. Natl. Acad. Sci. USA 93:9821-9826, (1996); U.S. Pat. No. 5,786,146.

Additional methylation detection methods include, but are not limited to, methylated CpG island amplification (see, Toyota et al., Cancer Res. 59:2307-12 (1999)) and those described in, e.g., U.S. Patent Publication 2005/0069879; Rein, et al. Nucleic Acids Res. 26 (10): 2255-64 (1998); Olek, et al. Nat Genet. 17 (3): 275-6 (1997); and PCT Publication No. WO 00/70090.

It is well known that methylation of genomic DNA can affect expression (transcription and/or translation) of nearby gene sequences. Therefore, in some embodiments, the methods include the step of correlating the methylation status of at least one cytosine in a DNA region with the expression of nearby coding sequences, as described in Tables 1-4 and in section “INFORMAL SEQUENCE LISTING.” For example, expression of gene sequences within about 1.0 kb, 1.5 kb, 2.0 kb, 2.5 kb, 3.0 kb, 3.5 kb or 4.0 kb in either the 3′ or 5′ direction from the cytosine of interest in the DNA region can be detected. Methods for measuring transcription and/or translation of a particular gene sequence are well known in the art. See, for example, Ausubel, Current Protocols in Molecular Biology, 1987-2006, John Wiley & Sons; and Sambrook and Russell, Molecular Cloning: A Laboratory Manual, 3rd Edition, 2000, Cold Spring Harbor Laboratory Press. In some embodiments, the gene or protein expression of a gene in Tables 1-4 and in section “INFORMAL SEQUENCE LISTING” is compared to a control, for example, the methylation status in the DNA region and/or the expression of a nearby gene sequence from a sample from an individual known to be negative for cancer or known to be positive for cancer, or to an expression level that distinguishes between cancer and noncancer states. Such methods, like the methods of detecting methylation described herein, are useful in providing diagnosis, prognosis, etc., of cancer.

In some embodiments, the methods further comprise the step of correlating the methylation status and expression of one or more of the gene regions identified in Tables 1-4 and in section “INFORMAL SEQUENCE LISTING.”

### IV. Cancer Detection

The present biomarkers and methods can be used in the diagnosis, prognosis, classification, prediction of disease risk, detection of recurrence of disease, and selection of treatment of a number of types of cancers. A cancer at any stage of progression can be detected, such as primary, metastatic, and recurrent cancers. Information regarding numerous types of cancer can be found, e.g., from the American Cancer Society (available on the worldwide web at cancer.org), or from, e.g., Harrison's Principles of Internal Medicine, Kaspar, et al., eds., 16th Edition, 2005, McGraw-Hill, Inc. Exemplary cancers that can be detected include lung, breast, renal, liver, ovarian, head and neck, thyroid, bladder, cervical, colon, endometrial, esophageal, prostate cancer or melanoma.

The present invention provides methods for determining whether or not a mammal (e.g., a human) has cancer, whether or not a biological sample taken from a mammal contains cancerous cells, estimating the risk or likelihood of a mammal developing cancer, classifying cancer types and stages, monitoring the efficacy of anti-cancer treatment, or selecting the appropriate anti-cancer treatment in a mammal with cancer. Such methods are based on the discovery that cancer cells have a different methylation status than normal cells in the DNA regions described in the invention. Accordingly, by determining whether or not a cell contains differentially methylated sequences in the DNA regions as described herein, it is possible to determine whether or not the cell is cancerous.

In numerous embodiments of the present invention, the presence of methylated nucleotides in the diagnostic biomarker sequences of the invention is detected in a biological sample, thereby detecting the presence or absence of cancerous cells in the biological sample.

In some embodiments, the biological sample comprises a tissue sample from a tissue suspected of containing cancerous cells. For example, in an individual suspected of having cancer, breast tissue, lymph tissue, lung tissue, brain tissue, or blood can be evaluated. Alternatively, lung, renal, liver, ovarian, head and neck, thyroid, bladder, cervical, colon, endometrial, esophageal, prostate, or skin tissue can be evaluated. The tissue or cells can be obtained by any method known in the art including, e.g., by surgery, biopsy, phlebotomy, swab, nipple discharge, stool, etc. In other embodiments, a tissue sample known to contain cancerous cells, e.g., from a tumor, will be analyzed for the presence or quantity of methylation at one or more of the diagnostic biomarkers of the invention to determine information about the cancer, e.g., the efficacy of certain treatments, the survival expectancy of the individual, etc. In some embodiments, the methods will be used in conjunction with additional diagnostic methods, e.g., detection of other cancer biomarkers, etc.

Genomic DNA samples can be obtained by any means known in the art. In cases where a particular phenotype or disease is to be detected, DNA samples should be prepared from a tissue of interest, or as appropriate, from blood. For example, DNA can be prepared from biopsy tissue to detect the methylation state of a particular locus associated with cancer. The nucleic acid-containing specimen used for detection of methylated loci (see, e.g., Ausubel et al., Current Protocols in Molecular Biology (1995 supplement)) may be from any source and may be extracted by a variety of techniques such as those described by Ausubel et al., Current Protocols in Molecular Biology (1995) or Sambrook et al., Molecular Cloning, A Laboratory Manual (3rd ed. 2001).

The methods of the invention can be used to evaluate individuals known or suspected to have cancer or as a routine clinical test, i.e., in an individual not necessarily suspected to have cancer. Further diagnostic assays can be performed to confirm the status of cancer in the individual.

Further, the present methods may be used to assess the efficacy of a course of treatment. For example, the efficacy of an anti-cancer treatment can be assessed by monitoring DNA methylation of the biomarker sequences described herein over time in a mammal having cancer. For example, a reduction or absence of methylation in any of the diagnostic biomarkers of the invention in a biological sample taken from a mammal following a treatment, compared to a level in a sample taken from the mammal before, or earlier in, the treatment, indicates efficacious treatment.

The methods detecting cancer can comprise the detection of one or more other cancer-associated polynucleotide or polypeptides sequences. Accordingly, detection of methylation of any one or more of the diagnostic biomarkers of the invention can be used either alone, or in combination with other biomarkers, for the diagnosis or prognosis of cancer.

The methods of the present invention can be used to determine the optimal course of treatment in a mammal with cancer. For example, the presence of methylated DNA within any of the diagnostic biomarkers of the invention or an increased quantity of methylation within any of the diagnostic biomarkers of the invention can indicate a reduced survival expectancy of a mammal with cancer, thereby indicating a more aggressive treatment for the mammal. In addition, a correlation can be readily established between the presence, absence or quantity of methylation at a diagnostic biomarker, as described herein, and the relative efficacy of one or another anti-cancer agent. Such analyses can be performed, e.g., retrospectively, i.e., by detecting methylation in one or more of the diagnostic genes in samples taken previously from mammals that have subsequently undergone one or more types of anti-cancer therapy, and correlating the known efficacy of the treatment with the presence, absence or levels of methylation of one or more of the diagnostic biomarkers.

In making a diagnosis, prognosis, risk assessment, classification, detection of recurrence or selection of therapy based on the presence or absence of methylation in at least one of the diagnostic biomarkers, the quantity of methylation may be compared to a threshold value that distinguishes between one diagnosis, prognosis, risk assessment, classification, etc., and another. For example, a threshold value can represent the degree of methylation found at a particular DNA region that adequately distinguishes between cancer samples and normal samples with a desired level of sensitivity and specificity. It is understood that a threshold value will likely vary depending on the assays used to measure methylation, but it is also understood that it is a relatively simple matter to determine a threshold value or range by measuring methylation of a DNA sequence in cancer samples and normal samples using the particular desired assay and then determining a value that distinguishes at least a majority of the cancer samples from a majority of non-cancer samples. If methylation of two or more DNA regions is detected, two or more different threshold values (one for each DNA region) will often, but not always, be used. Comparisons between a quantity of methylation of a sequence in a sample and a threshold value can be performed in any way known in the art. For example, a manual comparison can be made or a computer can compare and analyze the values to detect disease, assess the risk of contracting disease, determining a predisposition to disease, stage disease, diagnose disease, monitor, or aid in the selection of treatment for a person with disease.

In some embodiments, threshold values provide at least a specified sensitivity and specificity for detection of a particular cancer type. In some embodiments, the threshold value allows for at least a 50%, 60%, 70%, or 80% sensitivity and specificity for detection of a specific cancer, e.g., breast, lung, renal, liver, ovarian, head and neck, thyroid, bladder, cervical, colon, endometrial, esophageal, prostate cancer or melanoma.

In embodiments involving prognosis of cancer (including, for example, the prediction of progression of non-malignant lesions to invasive carcinoma, prediction of metastasis, prediction of disease recurrence or prediction of a response to a particular treatment), in some embodiments, the threshold value is set such that there is at least 10, 20, 30, 40, 50, 60, 70, 80% or more sensitivity and at least 70% specificity with regard to detecting cancer.

In some embodiments, the methods comprise recording a diagnosis, prognosis, risk assessment or classification, based on the methylation status determined from an individual. Any type of recordation is contemplated, including electronic recordation, e.g., by a computer.

### V. Kits

This invention also provides kits for the detection and/or quantification of the diagnostic biomarkers of the invention, or expression or methylation thereof using the methods described herein.

For kits that detect methylation, the kits of the invention can comprise at least one polynucleotide that hybridizes to at least one of the diagnostic biomarker sequences of the invention and at least one reagent for detection of gene methylation. Reagents for detection of methylation include, e.g., sodium bisulfite, polynucleotides designed to hybridize to sequence that is the product of a biomarker sequence of the invention if the biomarker sequence is not methylated (e.g., containing at least one C→U conversion), and/or a methylation-sensitive or methylation-dependent restriction enzyme. The kits can provide solid supports in the form of an assay apparatus that is adapted to use in the assay. The kits may further comprise detectable labels, optionally linked to a polynucleotide, e.g., a probe, in the kit. Other materials useful in the performance of the assays can also be included in the kits, including test tubes, transfer pipettes, and the like. The kits can also include written instructions for the use of one or more of these reagents in any of the assays described herein.

In some embodiments, the kits of the invention comprise one or more (e.g., 1, 2, 3, 4, or more) different polynucleotides (e.g., primers and/or probes) capable of specifically amplifying at least a portion of a DNA region where the DNA region is a sequence selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480. Optionally, one or more detectably-labeled polypeptides capable of hybridizing to the amplified portion can also be included in the kit. In some embodiments, the kits comprise sufficient primers to amplify 2, 3, 4, 5, 6, 7, 8, 9, 10, or more different DNA regions or portions thereof, and optionally include detectably-labeled polynucleotides capable of hybridizing to each amplified DNA region or portion thereof. The kits further can comprise a methylation-dependent or methylation sensitive restriction enzyme and/or sodium bisulfite.

In some embodiments, the kits comprise sodium bisulfite, primers and adapters (e.g., oligonucleotides that can be ligated or otherwise linked to genomic fragments) for whole genome amplification, and polynucleotides (e.g., detectably-labeled polynucleotides) to quantify the presence of the converted methylated and/or the converted unmethylated sequence of at least one cytosine from a DNA region that is selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480.

In some embodiments, the kits comprise a methylation sensing restriction enzymes (e.g., a methylation-dependent restriction enzyme and/or a methylation-sensitive restriction enzyme), primers and adapters for whole genome amplification, and polynucleotides to quantify the number of copies of at least a portion of a DNA region where the DNA region is selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480.

In some embodiments, the kits comprise a methylation binding moiety and one or more polynucleotides to quantify the number of copies of at least a portion of a DNA region where the DNA region is selected from the group consisting of SEQ ID NOS:385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479 and 480. A methylation binding moiety refers to a molecule (e.g., a polypeptide) that specifically binds to methyl-cytosine. Examples include restriction enzymes or fragments thereof that lack DNA cutting activity but retain the ability to bind methylated DNA, antibodies that specifically bind to methylated DNA, etc.).

### VI. Computer-Based Methods

The calculations for the methods described herein can involve computer-based calculations and tools. For example, a methylation value for a DNA region or portion thereof can be compared by a computer to a threshold value, as described herein. The tools are advantageously provided in the form of computer programs that are executable by a general purpose computer system (referred to herein as a “host computer”) of conventional design. The host computer may be configured with many different hardware components and can be made in many dimensions and styles (e.g., desktop PC, laptop, tablet PC, handheld computer, server, workstation, mainframe). Standard components, such as monitors, keyboards, disk drives, CD and/or DVD drives, and the like, may be included. Where the host computer is attached to a network, the connections may be provided via any suitable transport media (e.g., wired, optical, and/or wireless media) and any suitable communication protocol (e.g., TCP/IP); the host computer may include suitable networking hardware (e.g., modem, Ethernet card, WiFi card). The host computer may implement any of a variety of operating systems, including UNIX, Linux, Microsoft Windows, MacOS, or any other operating system.

Computer code for implementing aspects of the present invention may be written in a variety of languages, including PERL, C, C++, Java, JavaScript, VBScript, AWK, or any other scripting or programming language that can be executed on the host computer or that can be compiled to execute on the host computer. Code may also be written or distributed in low level languages such as assembler languages or machine languages.

The host computer system advantageously provides an interface via which the user controls operation of the tools. In the examples described herein, software tools are implemented as scripts (e.g., using PERL), execution of which can be initiated by a user from a standard command line interface of an operating system such as Linux or UNIX. Those skilled in the art will appreciate that commands can be adapted to the operating system as appropriate. In other embodiments, a graphical user interface may be provided, allowing the user to control operations using a pointing device. Thus, the present invention is not limited to any particular user interface.

Scripts or programs incorporating various features of the present invention may be encoded on various computer readable media for storage and/or transmission. Examples of suitable media include magnetic disk or tape, optical storage media such as compact disk (CD) or DVD (digital versatile disk), flash memory, and carrier signals adapted for transmission via wired, optical, and/or wireless networks conforming to a variety of protocols, including the Internet.

### EXAMPLES

### Example 1

### Identification of Cancer DNA Methylation Biomarkers and Design of Independent DNA Methylation Validation Assays

Loci that are differentially methylated in tumors relative to matched adjacent histologically normal tissue were identified using a DNA microarray-based technology platform that utilizes the methylation-dependent restriction enzyme McrBC. See, e.g., U.S. Pat. No. 7,186,512. The genomic region in which a given microarray feature can report DNA methylation status is dependent upon the molecular size of the DNA fragments that are labeled for the microarray hybridizations. In the microarray experiments, DNA in the size range of 1 to 4 kb was purified by agarose gel extraction and used as template for cyanogen dye labeling. Therefore, the genomic region interrogated by each microarray feature is 8 kb (i.e., 4 kb upstream and 4 bp downstream of the sequence represented by the microarray feature). Note that some features represent loci in which there is no Ensembl gene ID and no annotated transcribed gene within 1 kb of the microarray feature (e.g., Locus No.: 6, 22, 29, 31, 37, 46, 65, 71, and 96), and some features have Ensembl gene IDs but no gene description (e.g., Locus No.: 3, 11, 12, 18, 28, 36, 40, 41, 53, 56, 61, 67, 70, 76, 79, 84 and 88). Also note that some features represent loci in which more than one Ensembl annotated gene is within 1 kb of the microarray feature (e.g., Locus No.: 5, 23, 24, 36, 42, 49, 60, 62, 73, 75, 83, 88, 90, 92 and 94). DNA methylation at these loci may potentially affect the regulation of any of these neighboring genes. Detailed information about the selected loci can be found in Tables 1-4 and the section “INFORMAL SEQUENCE LISTING.”

PCR primers were designed that interrogated 96 total loci as follows: 36 loci which were predicted to be differentially methylated between breast tumor and histologically normal breast tissue, 36 loci which were predicted to be differentially methylated between lung tumor and histologically normal lung tissue, and 24 loci which were predicted to be differentially methylated between ovarian tumor and histologically normal ovarian tissue. Due to the functional properties of the enzyme, DNA methylation-dependent depletion of DNA fragments by McrBC is capable of monitoring the DNA methylation status of sequences neighboring the genomic sequences represented by the features on the microarray described above (wingspan). Since the size of DNA fragments analyzed as described in Example 1 was approximately 1-4 kb, an 8 kb region spanning the sequence represented by the microarray feature was selected as an estimate of the predicted region of differential methylation. For each locus, PCR primers were selected within an approximately 1 kb region flanking the genomic sequence represented on the DNA microarray (approximately 500 bp upstream and 500 bp downstream). Selection of primer sequences was guided by uniqueness of the primer sequence across the genome, as well as the distribution of purine-CG sequences within the 1 kb region. PCR primer pairs were selected to amplify an approximately 400-600 bp sequence within each 1 kb region. Optimal PCR cycling conditions for the primer pairs were empirically determined, and amplification of a specific PCR amplicon of the correct size was verified. The sequences of the microarray features, primer pairs and amplicons are indicated in Table 4, and in the “INFORMAL SEQUENCE LISTING” section.

TABLE 1
Features reporting differential DNA methylation between breast tumor and histologically
normal breast tissue and identity of annotated genes within 1 kb of each feature.

| Locus | | | |
|---|---|---|---|
| Number | Feature Name | Ensembl Gene ID | Annotation |
| 1 | ha1c_00037 | ENSG00000141646 | Mothers against decapentaplegic homolog 4 (SMAD 4) (Mothers |
| | | | against DPP homolog 4) (Deletion target in pancreatic carcinoma 4) |
| | | | (hSMAD4). [Source: Uniprot/SWISSPROT; Acc: Q13485] |
| 2 | ha1g_01283 | ENSG00000138650 | Protocadherin 10 precursor. |
| | | | [Source: Uniprot/SWISSPROT; Acc: Q9P2E7] |
| 3 | ha1g_01465 | ENSG00000184653 | no desc |
| 4 | ha1g_02335 | ENSG00000106006 | Homeobox protein Hox-A6 (Hox-1B). |
| | | | [Source: Uniprot/SWISSPROT; Acc: P31267] |
| 5 | ha1g_04114 | ENSG00000105808 | Ras GTPase-activating protein 4 (RasGAP-activating-like protein 2) |
| | | | (Calcium-promoted Ras inactivator). |
| | | | [Source: Uniprot/SWISSPROT; Acc: O43374] |
| | | ENSG00000170667 | Ras GTPase-activating protein 4 (RasGAP-activating-like protein 2) |
| | | | (Calcium-promoted Ras inactivator). |
| | | | [Source: Uniprot/SWISSPROT; Acc: O43374] |
| 6 | ha1g_04194 | N/A | N/A |
| 7 | ha1p_05922 | ENSG00000099256 | phosphoribosyl transferase domain containing 1 |
| | | | [Source: RefSeq_peptide; Acc: NP_064585] |
| 8 | ha1p_09663 | ENSG00000106511 | Homeobox protein MOX-2 (Mesenchyme homeobox 2) (Growth |
| | | | arrest-specific homeobox). |
| | | | [Source: Uniprot/SWISSPROT; Acc: P50222] |
| 9 | ha1p_100558 | ENSG00000174576 | HLH-PAS transcription factor NXF |
| | | | [Source: RefSeq_peptide; Acc: NP_849195] |
| 10 | ha1p_10286 | ENSG00000122691 | Twist-related protein 1 (H-twist). |
| | | | [Source: Uniprot/SWISSPROT; Acc: Q15672] |
| 11 | ha1p_108198 | ENSG00000179859 | no desc |
| 12 | ha1p_16916 | ENSG00000198317 | no desc |
| 13 | ha1p_18823 | ENSG00000112333 | Orphan nuclear receptor NR2E1 (Nuclear receptor TLX) (Tailless |
| | | | homolog) (Tll) (hTll). [Source: Uniprot/SWISSPROT; Acc: Q9Y466] |
| 14 | ha1p_22139 | ENSG00000118564 | F-box/LRR-repeat protein 5 (F-box and leucine-rich repeat protein |
| | | | 5) (F-box protein FBL4/FBL5) (p45SKP2-like protein). |
| | | | [Source: Uniprot/SWISSPROT; Acc: Q9UKA1] |
| 15 | ha1p_26420 | ENSG00000134371 | parafibromin [Source: RefSeq_peptide; Acc: NP_078805] |
| 16 | ha1p_38800 | ENSG00000130340 | Sorting nexin-9 (SH3 and PX domain-containing protein 1) (SDP1 |
| | | | protein). [Source: Uniprot/SWISSPROT; Acc: Q9Y5X1] |
| 17 | ha1p_41780 | ENSG00000163430 | Follistatin-related protein 1 precursor (Follistatin-like 1). |
| | | | [Source: Uniprot/SWISSPROT; Acc: Q12841] |
| 18 | ha1p_42103 | ENSG00000036054 | no desc |
| 19 | ha1p_47490 | ENSG00000179950 | fuse-binding protein-interacting represser isoform b |
| | | | [Source: RefSeq_peptide; Acc: NP_055096] |
| 20 | ha1p_47995 | ENSG00000179110 | Olfactory receptor 13C3. |
| | | | [Source: Uniprot/SWISSPROT; Acc: Q8NGS6] |
| 21 | ha1p_54181 | ENSG00000128602 | Smoothened homolog precursor (SMO) (Gx protein). |
| | | | [Source: Uniprot/SWISSPROT; Acc: Q99835] |
| 22 | ha1p_57326 | N/A | N/A |
| 23 | ha1p_60271 | ENSG00000163155 | LysM, putative peptidoglycan-binding, domain containing 1 |
| | | | [Source: RefSeq_peptide; Acc: NP_997716] |
| | | ENSG00000163156 | sodium channel modifier 1 isoform 1 |
| | | | [Source: RefSeq_peptide; Acc: NP_076946] |
| 24 | ha1p_62820 | ENSG00000174227 | GPI7 protein [Source: RefSeq_peptide; Acc: NP_060203] |
| | | ENSG00000186777 | no desc |
| 25 | ha1p_64271 | ENSG00000181449 | Transcription factor SOX-2. |
| | | | [Source: Uniprot/SWISSPROT; Acc: P48431] |
| 26 | ha1p_69412 | ENSG00000188015 | S100 calcium-binding protein A3 (S-100E protein). |
| | | | [Source: Uniprot/SWISSPROT; Acc: P33764] |
| 27 | ha1p_70432 | ENSG00000134020 | PEBP family protein precursor. |
| | | | [Source: Uniprot/SWISSPROT; Acc: Q96S96] |
| 28 | ha1p_71854 | ENSG00000160544 | no desc |
| 29 | ha1p_81638 | N/A | N/A |
| 30 | ha1p_86556 | ENSG00000165795 | NDRG2 protein (Syld709613 protein). |
| | | | [Source: Uniprot/SWISSPROT; Acc: Q9UN36] |
| 31 | ha1p_91110 | N/A | N/A |
| 32 | ha1p_94558 | ENSG00000128564 | Neurosecretory protein VGF precursor. |
| | | | [Source: Uniprot/SWISSPROT; Acc: O15240] |
| 33 | ha1p_96544 | ENSG00000187570 | Melanoma derived growth regulatory protein precursor (Melanoma |
| | | | inhibitory activity). [Source: Uniprot/SWISSPROT; Acc: Q16674] |
| 34 | ha1p_97458 | ENSG00000187800 | Novel protein similar to mouse Jedi soluble isoform 736 protein. |
| | | | [Source: Uniprot/SPTREMBL; Acc: Q5VY43] |
| 35 | ha1p_97786 | ENSG00000141750 | SH3 and cysteine rich domain 2 |
| | | | [Source: RefSeq_peptide; Acc: NP_945344] |
| 36 | ha1p_98401 | ENSG00000172803 | no desc |
| | | ENSG00000197847 | no desc |

TABLE 2
Features reporting differential DNA methylation between lung tumor and histologically
normal lung tissue and identity of annotated genes within 1 kb of each feature.

| Locus | | | |
|---|---|---|---|
| Number | Feature Name | Ensembl Gene ID | Annotation |
| 37 | ha1g_00353 | N/A | N/A |
| 38 | ha1p_00553 | ENSG00000088726 | transmembrane protein 40 |
| | | | [Source: RefSeq_peptide; Acc: NP_060776] |
| 39 | ha1p_04444 | ENSG00000151474 | FERM domain containing protein 4A. |
| | | | [Source: Uniprot/SWISSPROT; Acc: Q9P2Q2] |
| 40 | ha1p_07264 | ENSG00000109851 | no desc |
| 41 | ha1p_08159 | ENSG00000188590 | no desc |
| 42 | ha1p_103437 | ENSG00000161680 | no desc |
| | | ENSG00000161681 | Synaptotagmin-3 (Synaptotagmin III) (SytIII). |
| | | | [Source: Uniprot/SWISSPROT; Acc: Q9BQG1] |
| 43 | ha1p_105187 | ENSG00000108774 | Ras-related protein Rab-5C (RAB5L) (L1880). |
| | | | [Source: Uniprot/SWISSPROT; Acc: P51148] |
| 44 | ha1p_105778 | ENSG00000144218 | AF4/FMR2 family member 3 (LAF-4 protein) (Lymphoid nuclear |
| | | | protein related to AF4). |
| | | | [Source: Uniprot/SWISSPROT; Acc: P51826] |
| 45 | ha1p_10757 | ENSG00000197576 | Homeobox protein Hox-A4 (Hox-1D) (Hox-1.4). |
| | | | [Source: Uniprot/SWISSPROT; Acc: Q00056] |
| 46 | ha1p_108911 | N/A | N/A |
| 47 | ha1p_111312 | ENSG00000176130 | P2Y purinoceptor 11 (P2Y11). |
| | | | [Source: Uniprot/SWISSPROT; Acc: Q96G91] |
| 48 | ha1p_12483 | ENSG00000106554 | Coiled-coil-helix-coiled-coil-helix domain containing protein 3. |
| | | | [Source: Uniprot/SWISSPROT; Acc: Q9NX63] |
| 49 | ha1p_16097 | ENSG00000175879 | Homeobox protein Hox-D8 (Hox-4E) (Hox-5.4). |
| | | | [Source: Uniprot/SWISSPROT; Acc: P13378] |
| | | ENSG00000175892 | no desc |
| 50 | ha1p_27029 | ENSG00000162624 | LIM homeobox 8 [Source: RefSeq_peptide; Acc: NP_001001933] |
| 51 | ha1p_29823 | ENSG00000008197 | transcription factor AP-2 beta-like 1 |
| | | | [Source: RefSeq_peptide; Acc: NP_758438] |
| 52 | ha1p_40588 | ENSG00000135116 | Activator of apoptosis harakiri (Neuronal death protein DP5) |
| | | | (BH3 interacting domain protein 3). |
| | | | [Source: Uniprot/SWISSPROT; Acc: O00198] |
| 53 | ha1p_45692 | ENSG00000176147 | no desc |
| 54 | ha1p_47429 | ENSG00000054803 | Cerebellin 4 precursor (Cerebellin-like glycoprotein 1). |
| | | | [Source: Uniprot/SWISSPROT; Acc: Q9NTU7] |
| 55 | ha1p_49581 | ENSG00000099954 | Cat eye syndrome critical region protein 2. |
| | | | [Source: Uniprot/SWISSPROT; Acc: Q9BXF3] |
| 56 | ha1p_55371 | ENSG00000181384 | no desc |
| 57 | ha1p_58788 | ENSG00000108924 | Hepatic leukemia factor. |
| | | | [Source: Uniprot/SWISSPROT; Acc: Q16534] |
| 58 | ha1p_59216 | ENSG00000123576 | Extraembryonic, spermatogenesis, homeobox 1-like protein. |
| | | | [Source: Uniprot/SWISSPROT; Acc: Q8N693] |
| 59 | ha1p_61568 | ENSG00000196966 | Histone H3.1 (H3/a) (H3/c) (H3/d) (H3/f) (H3/h) (H3/i) (H3/j) |
| | | | (H3/k) (H3/l). [Source: Uniprot/SWISSPROT; Acc: P68431] |
| 60 | ha1p_61745 | ENSG00000115425 | Peroxisomal trans-2-enoyl-CoA reductase (EC 1.3.1.38) (TERP) |
| | | | (HPDHase) (pVI-ARL) (2,4-dienoyl-CoA reductase-related |
| | | | protein) (DCR-RP). [Source: Uniprot/SWISSPROT; Acc: Q9BY49] |
| | | ENSG00000163449 | no desc |
| 61 | ha1p_62060 | ENSG00000162877 | no desc |
| 62 | ha1p_62154 | ENSG00000158403 | None. [Source: Uniprot/SPTREMBL; Acc: Q92646] |
| | | ENSG00000178458 | Histone H3.1 (H3/a) (H3/c) (H3/d) (H3/f) (H3/h) (H3/i) (H3/j) |
| | | | (H3/k) (H3/l). [Source: Uniprot/SWISSPROT; Acc: P68431] |
| 63 | ha1p_62869 | ENSG00000100626 | Putative polypeptide N-acetylgalactosaminyltransferase-like protein |
| | | | 1 (EC 2.4.1.41) (Protein-UDP acetylgalactosaminyltransferase-like |
| | | | protein 1) (UDP-GalNAc:polypeptide N- |
| | | | acetylgalactosaminyltransferase- like protein 1) (Polypeptide |
| | | | GalNAc transferase-like |
| | | | [Source: Uniprot/SWISSPROT; Acc: Q8N428] |
| 64 | ha1p_64529 | ENSG00000157566 | Plasma glutathione peroxidase precursor (EC 1.11.1.9) (GSHPx-P) |
| | | | (Extracellular glutathione peroxidase) (GPx-P). |
| | | | [Source: Uniprot/SWISSPROT; Acc: P22352] |
| 65 | ha1p_77581 | N/A | N/A |
| 66 | ha1p_78965 | ENSG00000142700 | Doublesex-mab-3 (DM) domain (Fragment). |
| | | | [Source: Uniprot/SPTREMBL; Acc: Q96SC8] |
| 67 | ha1p_80400 | ENSG00000177107 | no desc |
| 68 | ha1p_81949 | ENSG00000135447 | Protein phosphatase inhibitor 1 (IPP-1) (1-1). |
| | | | [Source: Uniprot/SWISSPROT; Acc: Q13522] |
| 69 | ha1p_82549 | ENSG00000174059 | Hematopoietic progenitor cell antigen CD34 precursor. |
| | | | [Source: Uniprot/SWISSPROT; Acc: P28906] |
| 70 | ha1p_84580 | ENSG00000176938 | no desc |
| 71 | ha1p_86042 | N/A | N/A |
| 72 | ha1p_95305 | ENSG00000167889 | beta(1,6)-N-acetylglucosaminyltransferase V isoform 1 |
| | | | [Source: RefSeq_peptide; Acc: NP_653278] |

TABLE 3
Features reporting differential methylation between ovarian tumor and histologically
normal ovarian tissue and identity of annotated genes within 1 kb of each feature.

| Locus | | | |
|---|---|---|---|
| Number | Feature Name | Ensembl Gene ID | Annotation |
| 73 | CHR01P152508183 | ENSG00000160752 | Farnesyl pyrophosphate synthetase (FPP synthetase) (FPS) |
| | | | (Farnesyl diphosphate synthetase) [Includes: |
| | | | Dimethylallyltranstransferase (EC 2.5.1.1); Geranyltranstransferase |
| | | | (EC 2.5.1.10)]. [Source: Uniprot/SWISSPROT; Acc: P14324] |
| | | ENSG00000160753 | RUN and SH3 domain containing protein 1 (New molecule |
| | | | containing SH3 at the carboxy-terminus) (Nesca). |
| | | | [Source: Uniprot/SWISSPROT; Acc: Q9BVN2] |
| | | ENSG00000181363 | no desc |
| 74 | CHR02P046721735 | ENSG00000171142 | ATPase, H+ transporting, lysosomal 31 kDa, V1 subunit E isoform |
| | | | 2 [Source: RefSeq_peptide; Acc: NP_542384] |
| 75 | CHR04P001292657 | ENSG00000090316 | macrophage erythroblast attacher isoform 2 |
| | | | [Source: RefSeq_peptide; Acc: NP_005873] |
| | | ENSG00000188538 | no desc |
| 76 | CHR05P043085585 | ENSG00000177721 | no desc |
| 77 | CHR08P097127672 | ENSG00000156466 | growth differentiation factor 6 |
| | | | [Source: RefSeq_peptide; Acc: NP_001001557] |
| 78 | CHR08P102461728 | ENSG00000083307 | transcription factor CP2-like 3 |
| | | | [Source: RefSeq_peptide; Acc: NP_079191] |
| 79 | CHR08P143804195 | ENSG00000184865 | no desc |
| 80 | CHR09P021979668 | ENSG00000147889 | Cyclin-dependent kinase 4 inhibitor A (CDK4I) (p16-INK4) (p16- |
| | | | INK4a) (Multiple tumor suppressor 1) (MTS1). |
| | | | [Source: Uniprot/SWISSPROT; Acc: P42771] |
| 81 | CHR09P067743642 | ENSG00000107282 | Amyloid beta A4 precursor protein-binding family A member 1 |
| | | | (Neuron- specific X11 protein) (Neuronal Munc 18-1-interacting |
| | | | protein 1) (Mint- 1) (Adapter protein X11alpha). |
| | | | [Source: Uniprot/SWISSPROT; Acc: Q02410] |
| 82 | CHR11P010436241 | ENSG00000133805 | AMP deaminase 3 (EC 3.5.4.6) (AMP deaminase isoform E) |
| | | | (Erythrocyte AMP deaminase). |
| | | | [Source: Uniprot/SWISSPROT; Acc: Q01432] |
| 83 | CHR11P117233022 | ENSG00000137731 | Sodium/potassium-transporting ATPase gamma chain (Sodium |
| | | | pump gamma chain) (Na+/K+ ATPase gamma subunit) (FXYD |
| | | | domain-containing ion transport regulator 2). |
| | | | [Source: Uniprot/SWISSPROT; Acc: P54710] |
| | | ENSG00000137746 | no desc |
| 84 | CHR12P044081945 | ENSG00000177119 | no desc |
| 85 | CHR13P042532794 | ENSG00000139656 | MGC5590 protein. [Source: Uniprot/SPTREMBL; Acc: Q9BVW6] |
| 86 | CHR14P049549993 | ENSG00000100505 | Tripartite motif protein 9 (RING finger protein 91). |
| | | | [Source: Uniprot/SWISSPROT; Acc: Q9C026] |
| 87 | CHR15P062682028 | ENSG00000180357 | zinc finger protein 609 [Source: RefSeq_peptide; Acc: NP_055857] |
| 88 | CHR16P070471895 | ENSG00000132613 | no desc |
| | | ENSG00000183452 | no desc |
| 89 | CHR17P007309455 | ENSG00000132535 | Postsynaptic density protein 95 (PSD-95) (Synapse-associated |
| | | | protein 90) (SAP90) (Discs large homolog 4). |
| | | | [Source: Uniprot/SWISSPROT; Acc: P78352] |
| 90 | CHR19P047620296 | ENSG00000079435 | Hormone-sensitive lipase (EC 3.1.1.79) (HSL). |
| | | | [Source: Uniprot/SWISSPROT; Acc: Q05469] |
| | | ENSG00000182797 | no desc |
| 91 | CHR19P054350430 | ENSG00000130528 | Sarcoplasmic reticulum histidine-rich calcium-binding protein |
| | | | precursor. [Source: Uniprot/SWISSPROT; Acc: P23327] |
| 92 | CHR19P059796623 | ENSG00000104974 | Leukocyte immunoglobulin-like receptor subfamily A member 1 |
| | | | precursor (Leucocyte immunoglobulin-like receptor 6) (LIR-6) |
| | | | (CD85i antigen). [Source: Uniprot/SWISSPROT; Acc: O75019] |
| | | ENSG00000131042 | Leukocyte immunoglobulin-like receptor subfamily B member 2 |
| | | | precursor (Leukocyte immunoglobulin-like receptor 2) (LIR-2) |
| | | | (Immunoglobulin- like transcript 4) (ILT-4) |
| | | | (Monocyte/macrophage immunoglobulin-like receptor 10) (MIR- |
| | | | 10) (CD85d antigen).[Source: Uniprot/SWISSPROT; Acc: O75019] |
| 93 | CHR20P038041321 | ENSG00000101438 | Vesicular inhibitory amino acid transporter (GABA and glycine |
| | | | transporter) (Vesicular GABA transporter) (hVIAAT) (Solute |
| | | | carrier family 32 member 1). |
| | | | [Source: Uniprot/SWISSPROT; Acc: Q9H598] |
| 94 | ha1p_108204_150 | ENSG00000170043 | Trafficking protein particle complex subunit 1 (BET5 homolog) |
| | | | (Multiple myeloma protein 2) (MUM-2). |
| | | | [Source: Uniprot/SWISSPROT; Acc: Q9Y5R8] |
| | | ENSG00000170049 | Voltage-gated potassium channel beta-3 subunit (K(+) channel |
| | | | beta-3 subunit) (Kv-beta-3). |
| | | | [Source: Uniprot/SWISSPROT; Acc: O43448] |
| 95 | ha1p_48631_150 | ENSG00000124839 | Ras-related protein Rab-17. |
| | | | [Source: Uniprot/SWISSPROT; Acc: Q9H0T7] |
| 96 | ha1p_94692_150 | N/A | N/A |

TABLE 4
Sequence identifier numbers (SEQ ID NOS:) for all sequences described
in the application. See section “INFORMAL SEQUENCE LISTING”
for actual sequences as listed by number in the table.

| | | | | | DNA |
|---|---|---|---|---|---|
| | Locus | Left | Right | Amplicon | Region |
| Feature Name | Number | primer | primer | Sequence | Sequence |
| ha1c_00037 | 1 | 97 | 98 | 289 | 385 |
| ha1g_01283 | 2 | 99 | 100 | 290 | 386 |
| ha1g_01465 | 3 | 101 | 102 | 291 | 387 |
| ha1g_02335 | 4 | 103 | 104 | 292 | 388 |
| ha1g_04114 | 5 | 105 | 106 | 293 | 389 |
| ha1g_04194 | 6 | 107 | 108 | 294 | 390 |
| ha1p_05922 | 7 | 109 | 110 | 295 | 391 |
| ha1p_09663 | 8 | 111 | 112 | 296 | 392 |
| ha1p_100558 | 9 | 113 | 114 | 297 | 393 |
| ha1p_10286 | 10 | 115 | 116 | 298 | 394 |
| ha1p_108198 | 11 | 117 | 118 | 299 | 395 |
| ha1p_16916 | 12 | 119 | 120 | 300 | 396 |
| ha1p_18823 | 13 | 121 | 122 | 301 | 397 |
| ha1p_22139 | 14 | 123 | 124 | 302 | 398 |
| ha1p_26420 | 15 | 125 | 126 | 303 | 399 |
| ha1p_38800 | 16 | 127 | 128 | 304 | 400 |
| ha1p_41780 | 17 | 129 | 130 | 305 | 401 |
| ha1p_42103 | 18 | 131 | 132 | 306 | 402 |
| ha1p_47490 | 19 | 133 | 134 | 307 | 403 |
| ha1p_47995 | 20 | 135 | 136 | 308 | 404 |
| ha1p_54181 | 21 | 137 | 138 | 309 | 405 |
| ha1p_57326 | 22 | 139 | 140 | 310 | 406 |
| ha1p_60271 | 23 | 141 | 142 | 311 | 407 |
| ha1p_62820 | 24 | 143 | 144 | 312 | 408 |
| ha1p_64271 | 25 | 145 | 146 | 313 | 409 |
| ha1p_69412 | 26 | 147 | 148 | 314 | 410 |
| ha1p_70432 | 27 | 149 | 150 | 315 | 411 |
| ha1p_71854 | 28 | 151 | 152 | 316 | 412 |
| ha1p_81638 | 29 | 153 | 154 | 317 | 413 |
| ha1p_86556 | 30 | 155 | 156 | 318 | 414 |
| ha1p_91110 | 31 | 157 | 158 | 319 | 415 |
| ha1p_94558 | 32 | 159 | 160 | 320 | 416 |
| ha1p_96544 | 33 | 161 | 162 | 321 | 417 |
| ha1p_97458 | 34 | 163 | 164 | 322 | 418 |
| ha1p_97786 | 35 | 165 | 166 | 323 | 419 |
| ha1p_98401 | 36 | 167 | 168 | 324 | 420 |
| ha1g_00353 | 37 | 169 | 170 | 325 | 421 |
| ha1p_00553 | 38 | 171 | 172 | 326 | 422 |
| ha1p_04444 | 39 | 173 | 174 | 327 | 423 |
| ha1p_07264 | 40 | 175 | 176 | 328 | 424 |
| ha1p_08159 | 41 | 177 | 178 | 329 | 425 |
| ha1p_103437 | 42 | 179 | 180 | 330 | 426 |
| ha1p_105187 | 43 | 181 | 182 | 331 | 427 |
| ha1p_105778 | 44 | 183 | 184 | 332 | 428 |
| ha1p_10757 | 45 | 185 | 186 | 333 | 429 |
| ha1p_108911 | 46 | 187 | 188 | 334 | 430 |
| ha1p_111312 | 47 | 189 | 190 | 335 | 431 |
| ha1p_12483 | 48 | 191 | 192 | 336 | 432 |
| ha1p_16097 | 49 | 193 | 194 | 337 | 433 |
| ha1p_27029 | 50 | 195 | 196 | 338 | 434 |
| ha1p_29823 | 51 | 197 | 198 | 339 | 435 |
| ha1p_40588 | 52 | 199 | 200 | 340 | 436 |
| ha1p_45692 | 53 | 201 | 202 | 341 | 437 |
| ha1p_47429 | 54 | 203 | 204 | 342 | 438 |
| ha1p_49581 | 55 | 205 | 206 | 343 | 439 |
| ha1p_55371 | 56 | 207 | 208 | 344 | 440 |
| ha1p_58788 | 57 | 209 | 210 | 345 | 441 |
| ha1p_59216 | 58 | 211 | 212 | 346 | 442 |
| ha1p_61568 | 59 | 213 | 214 | 347 | 443 |
| ha1p_61745 | 60 | 215 | 216 | 348 | 444 |
| ha1p_62060 | 61 | 217 | 218 | 349 | 445 |
| ha1p_62154 | 62 | 219 | 220 | 350 | 446 |
| ha1p_62869 | 63 | 221 | 222 | 351 | 447 |
| ha1p_64529 | 64 | 223 | 224 | 352 | 448 |
| ha1p_77581 | 65 | 225 | 226 | 353 | 449 |
| ha1p_78965 | 66 | 227 | 228 | 354 | 450 |
| ha1p_80400 | 67 | 229 | 230 | 355 | 451 |
| ha1p_81949 | 68 | 231 | 232 | 356 | 452 |
| ha1p_82549 | 69 | 233 | 234 | 357 | 453 |
| ha1p_84580 | 70 | 235 | 236 | 358 | 454 |
| ha1p_86042 | 71 | 237 | 238 | 359 | 455 |
| ha1p_95305 | 72 | 239 | 240 | 360 | 456 |
| CHR01P152508183 | 73 | 241 | 242 | 361 | 457 |
| CHR02P046721735 | 74 | 243 | 244 | 362 | 458 |
| CHR04P001292657 | 75 | 245 | 246 | 363 | 459 |
| CHR05P043085585 | 76 | 247 | 248 | 364 | 460 |
| CHR08P097127672 | 77 | 249 | 250 | 365 | 461 |
| CHR08P102461728 | 78 | 251 | 252 | 366 | 462 |
| CHR08P143804195 | 79 | 253 | 254 | 367 | 463 |
| CHR09P021979668 | 80 | 255 | 256 | 368 | 464 |
| CHR09P067743642 | 81 | 257 | 258 | 369 | 465 |
| CHR11P010436241 | 82 | 259 | 260 | 370 | 466 |
| CHR11P117233022 | 83 | 261 | 262 | 371 | 467 |
| CHR12P044081945 | 84 | 263 | 264 | 372 | 468 |
| CHR13P042532794 | 85 | 265 | 266 | 373 | 469 |
| CHR14P049549993 | 86 | 267 | 268 | 374 | 470 |
| CHR15P062682028 | 87 | 269 | 270 | 375 | 471 |
| CHR16P070471895 | 88 | 271 | 272 | 376 | 472 |
| CHR17P007309455 | 89 | 273 | 274 | 377 | 473 |
| CHR19P047620296 | 90 | 275 | 276 | 378 | 474 |
| CHR19P054350430 | 91 | 277 | 278 | 379 | 475 |
| CHR19P059796623 | 92 | 279 | 280 | 380 | 476 |
| CHR20P038041321 | 93 | 281 | 282 | 381 | 477 |
| ha1p_108204_150 | 94 | 283 | 284 | 382 | 478 |
| ha1p_48631_150 | 95 | 285 | 286 | 383 | 479 |
| ha1p_94692_150 | 96 | 287 | 288 | 384 | 480 |

### Example 2

### Validation of DNA Methylation Changes in a Large Number of Independent Breast Tumor and Histologically Normal Breast Samples

The differential DNA methylation status of 36 loci (Table 1) was further validated by analyzing an independent panel of 24 breast carcinoma samples and 25 histologically normal breast samples. Each sample was split into two equal portions of 4 μg. One portion was digested with McrBC (Treated Portion) in a total volume of 200 μL including 1×NEB2 buffer (New England Biolabs), 0.1 mg/mL bovine serum albumin (New England Biolabs), 2 mM GTP (Roche) and 32 units McrBC (New England Biolabs). The second portion was mock treated under identical conditions, except that 3.2 μL sterile 50% glycerol was added instead of McrBC enzyme (Untreated Portion). Samples were incubated at 37° C. for approximately 12 hours, followed by incubation at 60° C. to inactivate the McrBC enzyme.

The extent of McrBC cleavage at each locus was monitored by quantitative real-time PCR (qPCR). For each assayed locus, qPCR was performed using 20 ng of the Untreated Portion DNA as template and, separately, using 20 ng of the Treated Portion DNA as template. Each reaction was performed in 10 μL total volume including 1× LightCycler 480 SYBR Green I Master mix (Roche) and 625 nM of each primer. Reactions were run in a Roche LightCycler 480 instrument. Cycling conditions were: 95° C. for 5 min.; 45 cycles of 95° C. for 1 min., 66° C. for 30 sec., 72° C. for 1 min., 83° C. for 2 sec. followed by a plate read. Melting curves were calculated under the following conditions: 95° C. for 5 sec., 65° C. for 1 min., 65° C. to 95° C. at 2.5° C./sec. ramp rate with continuous plate reads. Each Untreated/Treated qPCR reaction pair was performed in duplicate. The difference in the cycle number at which amplification crossed threshold (delta Ct) was calculated for each Untreated/Treated qPCR reaction pair by subtracting the Ct of the Untreated Portion from the Ct of the Treated Portion. Because McrBC-mediated cleavage between the two primers increases the Ct of the Treated Portion, increasing delta Ct values reflect increasing measurements of local DNA methylation densities. The average delta Ct between the two replicate Untreated/Treated qPCR reactions was calculated, as well as the standard deviation between the two delta Ct values.

Table 5 indicates the percent sensitivity and specificity for each locus. Gain biomarkers are biomarkers that show more methylation in tumor samples than normal samples and loss biomarkers show conversely. For gain biomarkers, sensitivity reflects the frequency of scoring a known tumor sample as positive for DNA methylation at each locus while specificity reflects the frequency of scoring a known normal sample as negative for DNA methylation at each locus. For loss biomarkers, sensitivity reflects the frequency of scoring a known tumor sample as negative for DNA methylation at each locus while specificity reflects the frequency of scoring a known normal sample as positive for DNA methylation at each locus. Receiver-operator characteristic analysis (Lasko, et al. (2005) Journal of Biomedical Informatics 38 (5):404-415.) was used to determine empirical threshold values for classifying tissue samples. The analysis was performed independently for each locus. Percent sensitivity of gain biomarkers was calculated as the number of tumor samples with an average delta Ct greater than the threshold divided by the total number of tumor samples analyzed for that locus (i.e., excluding any measurements with a standard deviation between qPCR replicates >1 cycle)×100. Percent specificity of gain biomarkers was calculated as (1−(the number of normal samples with an average delta Ct greater than the threshold divided by the total number of normal samples analyzed for that locus))×100. The sensitivity and specificity of loss biomarkers was calculated using the number of samples below the threshold. Resulting sensitivity and specificity calculations are shown in Table 5. The sensitivity and specificity of the differential DNA methylation status of any given locus may be increased by further optimization of the precise local genetic region interrogated by a DNA methylation-sensing assay.

TABLE 5
Sensitivity and specificity of differentially methylated loci in a panel of 24
breast tumor samples and 25 histologically normal breast samples.

| Locus | | | | Difference in | | |
|---|---|---|---|---|---|---|
| Number | Feature Name | Type | Threshold | Medians (T − N) | Sensitivity | Specificity |
| 1 | ha1c_00037 | Gain | 1.995 | 1.255 | 69.57% | 96.00% |
| 2 | ha1g_01283 | Gain | 0.555 | 0.235 | 33.33% | 95.83% |
| 3 | ha1g_01465 | Gain | 0.54 | 0 | 20.83% | 96.00% |
| 4 | ha1g_02335 | Gain | 1.87 | 1.625 | 75.00% | 92.00% |
| 5 | ha1g_04114 | Gain | 1.18 | 0 | 4.17% | 95.83% |
| 6 | ha1g_04194 | Gain | 0.515 | 1.16 | 79.17% | 95.83% |
| 7 | ha1p_05922 | Gain | 1.495 | 1.725 | 91.67% | 100.00% |
| 8 | ha1p_09663 | Gain | 1.205 | 0.6025 | 50.00% | 100.00% |
| 9 | ha1p_100558 | Gain | 0.805 | 0.36 | 33.33% | 91.67% |
| 10 | ha1p_10286 | Gain | 0.505 | 0.6675 | 66.67% | 92.00% |
| 11 | ha1p_108198 | Gain | 1.905 | 2.08 | 87.50% | 100.00% |
| 12 | ha1p_16916 | Loss | 3.445 | −2.3925 | 79.17% | 90.91% |
| 13 | ha1p_18823 | Gain | 0.605 | 0.155 | 23.81% | 90.91% |
| 14 | ha1p_22139 | Gain | 1.405 | 0.5475 | 45.83% | 90.91% |
| 15 | ha1p_26420 | Gain | 2.01 | 0.43 | 45.45% | 100.00% |
| 16 | ha1p_38800 | Gain | 2.52 | 0.905 | 66.67% | 92.00% |
| 17 | ha1p_41780 | Gain | 4.56 | 1.7675 | 77.27% | 90.91% |
| 18 | ha1p_42103 | Gain | 0.615 | 0 | 4.17% | 96.00% |
| 19 | ha1p_47490 | Gain | 1.18 | 2.9925 | 87.50% | 88.00% |
| 20 | ha1p_47995 | Loss | 4.28 | −1.9925 | 83.33% | 72.00% |
| 21 | ha1p_54181 | Gain | 0.715 | 0 | 22.22% | 100.00% |
| 22 | ha1p_57326 | Gain | 1.355 | 1.245 | 83.33% | 92.00% |
| 23 | ha1p_60271 | Gain | 0.59 | 0.155 | 50.00% | 88.89% |
| 24 | ha1p_62820 | Gain | 3.66 | 0.585 | 50.00% | 100.00% |
| 25 | ha1p_64271 | Gain | 0.51 | 0 | 20.83% | 100.00% |
| 26 | ha1p_69412 | Gain | 2.285 | 1.8275 | 91.67% | 90.91% |
| 27 | ha1p_70432 | Gain | 2.29 | 1.475 | 73.68% | 100.00% |
| 28 | ha1p_71854 | Gain | 3.335 | 1.0575 | 84.21% | 77.78% |
| 29 | ha1p_81638 | Gain | 0.555 | 0 | 20.83% | 100.00% |
| 30 | ha1p_86556 | Gain | 0.62 | 0.4075 | 55.00% | 95.00% |
| 31 | ha1p_91110 | Loss | 4.835 | −2.775 | 75.00% | 100.00% |
| 32 | ha1p_94558 | Gain | 1.485 | 0.5175 | 81.82% | 60.87% |
| 33 | ha1p_96544 | Gain | 3.36 | 2.09 | 53.85% | 100.00% |
| 34 | ha1p_97458 | Gain | 1.72 | 0.145 | 37.50% | 100.00% |
| 35 | ha1p_97786 | Gain | 0.625 | 0.32 | 46.15% | 87.50% |
| 36 | ha1p_98401 | Gain | 0.67 | 0.7775 | 62.50% | 96.00% |

### Example 3

### Validation of DNA Methylation Changes in a Large Number of Independent Lung Tumor and Histologically Normal Lung Samples

The differential DNA methylation status of 36 loci was further validated by analyzing an independent panel of 25 lung carcinoma and 35 histologically normal lung tissue samples. Each sample was split into two equal portions of 4 μg. One portion was digested with McrBC (Treated Portion) in a total volume of 200 μL including 1×NEB2 buffer (New England Biolabs), 0.1 mg/mL bovine serum albumin (New England Biolabs), 2 mM GTP (Roche) and 32 units McrBC (New England Biolabs). The second portion was mock treated under identical conditions, except that 3.2 μL sterile 50% glycerol was added instead of McrBC enzyme (Untreated Portion). Samples were incubated at 37° C. for approximately 12 hours, followed by incubation at 60° C. to inactivate the McrBC enzyme. qPCR reactions and data analysis were performed as described in Example 2.

Sensitivity and specificity were calculated using ROC analysis derived thresholds as described above. Table 6 indicates the percent sensitivity and specificity for each locus.

TABLE 6
Sensitivity and specificity of differentially methylated loci in a panel of
25 lung tumor samples and 35 histologically normal lung samples.

| Locus | | | | Difference in | | |
|---|---|---|---|---|---|---|
| Number | Feature Name | Type | Threshold | Medians (T − N) | Sensitivity | Specificity |
| 37 | ha1g_00353 | Gain | 0.5 | 0.11 | 36.00% | 97.14% |
| 38 | ha1p_00553 | Loss | 1.36 | −0.115 | 86.36% | 45.71% |
| 39 | ha1p_04444 | Gain | 1.2 | 0.225 | 52.17% | 79.41% |
| 40 | ha1p_07264 | Gain | 0.56 | 0.21 | 48.00% | 100.00% |
| 41 | ha1p_08159 | Loss | 5.45 | −2.95 | 96.00% | 65.71% |
| 42 | ha1p_103437 | Loss | 5.44 | −2.94 | 80.00% | 82.86% |
| 43 | ha1p_105187 | Gain | 1.76 | 0.555 | 72.00% | 88.57% |
| 44 | ha1p_105778 | Gain | 2.58 | 0.475 | 56.00% | 85.29% |
| 45 | ha1p_10757 | Gain | 1.1 | 1.495 | 76.00% | 91.43% |
| 46 | ha1p_108911 | Loss | 1.36 | −0.425 | 60.00% | 88.57% |
| 47 | ha1p_111312 | Loss | 1.37 | −0.73 | 88.00% | 94.12% |
| 48 | ha1p_12483 | Gain | 2.65 | 0.115 | 32.00% | 88.57% |
| 49 | ha1p_16097 | Gain | 0.5 | 0 | 32.00% | 94.29% |
| 50 | ha1p_27029 | Gain | 0.69 | 0.61 | 54.17% | 88.57% |
| 51 | ha1p_29823 | Gain | 0.6 | 0.655 | 60.00% | 94.29% |
| 52 | ha1p_40588 | Gain | 0.62 | 0.33 | 48.00% | 85.29% |
| 53 | ha1p_45692 | Loss | 3.53 | −1.51 | 86.96% | 90.91% |
| 54 | ha1p_47429 | Gain | 1.04 | 0.665 | 68.00% | 97.14% |
| 55 | ha1p_49581 | Gain | 0.82 | 0.39 | 68.00% | 88.57% |
| 56 | ha1p_55371 | Loss | 0.83 | −0.56 | 84.00% | 51.43% |
| 57 | ha1p_58788 | Gain | 0.63 | 0.57 | 44.00% | 97.14% |
| 58 | ha1p_59216 | Gain | 2.07 | 0.01 | 17.39% | 100.00% |
| 59 | ha1p_61568 | Gain | 1.65 | 1.375 | 50.00% | 96.43% |
| 60 | ha1p_61745 | Loss | 2.31 | −0.88 | 60.87% | 80.00% |
| 61 | ha1p_62060 | Gain | 1.99 | 0.75 | 54.17% | 88.57% |
| 62 | ha1p_62154 | Gain | 0.83 | 0.29 | 52.00% | 91.43% |
| 63 | ha1p_62869 | Gain | 0.54 | 0.315 | 52.00% | 88.57% |
| 64 | ha1p_64529 | Gain | 4.27 | 1.865 | 91.30% | 75.76% |
| 65 | ha1p_77581 | Gain | 0.6 | 0.725 | 62.50% | 97.14% |
| 66 | ha1p_78965 | Gain | 0.5 | 0.32 | 37.50% | 94.29% |
| 67 | ha1p_80400 | Gain | 5.95 | 0 | 88.00% | 25.71% |
| 68 | ha1p_81949 | Gain | 0.87 | 0.38 | 62.50% | 74.29% |
| 69 | ha1p_82549 | Gain | 1.06 | 0.16 | 44.00% | 82.86% |
| 70 | ha1p_84580 | Loss | 1.32 | −0.93 | 72.00% | 88.24% |
| 71 | ha1p_86042 | Loss | 1.11 | 0.09 | 100.00% | 24.24% |
| 72 | ha1p_95305 | Loss | 3.94 | −1.655 | 84.00% | 82.86% |

### Example 4

### Validation of DNA Methylation Changes in a Large Number of Independent Ovarian Tumor and Histologically Normal Ovarian Samples

The differential DNA methylation status of 24 loci was further validated by analyzing an independent panel of 23 ovarian carcinoma and 25 histologically normal ovarian tissue samples. The normal ovarian tissues included in this panel were obtained from oophorectomies unrelated to ovarian cancer. Each sample was split into two equal portions of 4 μg. One portion was digested with McrBC (Treated Portion) in a total volume of 200 μL including 1×NEB2 buffer (New England Biolabs), 0.1 mg/mL bovine serum albumin (New England Biolabs), 2 mM GTP (Roche) and 32 units McrBC (New England Biolabs). The second portion was mock treated under identical conditions, except that 3.2 μL sterile 50% glycerol was added instead of McrBC enzyme (Untreated Portion). Samples were incubated at 37° C. for approximately 12 hours, followed by incubation at 60° C. to inactivate the McrBC enzyme. qPCR reactions and data analysis were performed as described in Example 2.

Sensitivity and specificity were calculated using ROC analysis derived thresholds as described above. Table 7 indicates the percent sensitivity and specificity for each locus.

TABLE 7
Sensitivity and specificity of differentially methylated loci in a panel of 23
ovarian tumor samples and 25 histologically normal ovarian samples.

| Locus | | | | Difference in | | |
|---|---|---|---|---|---|---|
| Number | Feature Name | Type | Threshold | Medians (T − N) | Sensitivity | Specificity |
| 73 | CHR01P152508183 | Gain | 2.29 | 0.65 | 60.87% | 65.22% |
| 74 | CHR02P046721735 | Gain | 2.86 | 2.495 | 73.91% | 96.00% |
| 75 | CHR04P001292657 | Loss | 4.92 | −1.3025 | 63.64% | 84.00% |
| 76 | CHR05P043085585 | Loss | 0.54 | −0.895 | 95.65% | 76.00% |
| 77 | CHR08P097127672 | Gain | 0.745 | 0.3675 | 50.00% | 95.83% |
| 78 | CHR08P102461728 | Loss | 2.595 | −4.415 | 95.45% | 100.00% |
| 79 | CHR08P143804195 | Gain | 5.56 | 1.67 | 52.17% | 92.00% |
| 80 | CHR09P021979668 | Gain | 2.39 | 0.915 | 52.17% | 95.65% |
| 81 | CHR09P067743642 | Gain | 0.85 | 0.935 | 59.09% | 84.00% |
| 82 | CHR11P010436241 | Loss | 5.335 | −3.61 | 90.91% | 84.00% |
| 83 | CHR11P117233022 | Gain | 1.39 | 0.695 | 56.52% | 82.61% |
| 84 | CHR12P044081945 | Gain | 3.4 | 0.94 | 45.45% | 96.00% |
| 85 | CHR13P042532794 | Gain | 3.045 | 0.005 | 30.43% | 91.30% |
| 86 | CHR14P049549993 | Loss | 1.72 | −0.22 | 84.62% | 41.18% |
| 87 | CHR15P062682028 | Gain | 3.185 | 2.1875 | 80.95% | 77.27% |
| 88 | CHR16P070471895 | Loss | 0.855 | 0.07 | 66.67% | 42.86% |
| 89 | CHR17P007309455 | Loss | 0.53 | −0.4075 | 66.67% | 66.67% |
| 90 | CHR19P047620296 | Gain | 1.78 | 1.4125 | 73.91% | 75.00% |
| 91 | CHR19P054350430 | Gain | 2.19 | 0.455 | 69.57% | 60.87% |
| 92 | CHR19P059796623 | Loss | 4.96 | −3.075 | 91.30% | 79.17% |
| 93 | CHR20P038041321 | Gain | 0.55 | 0.325 | 43.48% | 100.00% |
| 94 | ha1p_108204_150 | Gain | 0.87 | 0.875 | 54.55% | 92.00% |
| 95 | ha1p_48631_150 | Loss | 5.855 | −4.595 | 90.91% | 95.83% |
| 96 | ha1p_94692_150 | Gain | 2.13 | 1.4975 | 55.00% | 90.48% |

### Example 5

### Analysis of Loci Discovered to be Differentially DNA Methylated in Breast Cancer Among Lung and Ovarian Tumor and Histologically Normal Samples

The differential DNA methylation status of 36 loci found to be differentially DNA methylated in breast tumors relative to histologically normal breast samples (Table 5) was monitored in a randomly selected panel of 10 lung tumor samples and 10 histologically normal lung samples (Table 8). The same loci were analyzed in a randomly selected panel of 10 ovarian tumor samples and 10 histologically normal ovary samples (Table 9). Each sample was split into two equal portions of 3 μg. One portion was digested with McrBC (Treated Portion) in a total volume of 150 μL including 1×NEB2 buffer (New England Biolabs), 0.1 mg/mL bovine serum albumin (New England Biolabs), 2 mM GTP (Roche) and 48 units McrBC (New England Biolabs). The second portion was mock treated under identical conditions, except that 4.8 μL sterile 50% glycerol was added instead of McrBC enzyme (Untreated Portion). Samples were incubated at 37° C. for approximately 12 hours, followed by incubation at 60° C. to inactivate the McrBC enzyme. qPCR reactions and data analysis were performed as described in Example 2.

Sensitivity and specificity were calculated using ROC analysis derived thresholds as described above. Table 8 indicates the percent sensitivity and specificity for each locus analyzed in the panel of lung tumor and histologically normal lung samples. Table 9 indicates the percent sensitivity and specificity for each locus analyzed in the panel of ovarian tumor and histologically normal ovary samples. The number of samples scoring as positive for the methylation change among the analyzed tumor samples is indicated (Sensitivity (n of n)). For example, “7 of 10” indicates that seven tumor samples scored in the positive range as determined by ROC based average dCt thresholds (Threshold) among a total of 10 tumor samples analyzed. The number of samples scoring as negative for the methylation change among the analyzed histologically normal samples is also indicated (Specificity (n of n)).

TABLE 8
Sensitivity and specificity of loci identified as differentially methylated in breast tumors among
a panel of 10 lung tumor samples and 10 histologically normal lung samples.

| | | | | Difference | | | | |
|---|---|---|---|---|---|---|---|---|
| Locus | Feature | | | in Medians | | | Sensitivity | Specificity |
| Number | Name | Type | Threshold | (T − N) | Sensitivity | Specificity | (n of n) | (n of n) |
| 1 | ha1c_00037 | Gain | 3.155 | 0.7375 | 70.00% | 100.00% | 7 of 10 | 10 of 10 |
| 2 | ha1g_01283 | Gain | 0.58 | 0.505 | 90.00% | 100.00% | 9 of 10 | 10 of 10 |
| 3 | ha1g_01465 | Gain | 0.555 | 0.34 | 50.00% | 100.00% | 5 of 10 | 10 of 10 |
| 4 | ha1g_02335 | Gain | 1.985 | 0.875 | 60.00% | 100.00% | 6 of 10 | 10 of 10 |
| 5 | ha1g_04114 | Gain | 0.57 | 0.16 | 20.00% | 100.00% | 2 of 10 | 10 of 10 |
| 6 | ha1g_04194 | Gain | 1.47 | 0.7275 | 80.00% | 100.00% | 8 of 10 | 10 of 10 |
| 7 | ha1p_05922 | Gain | 3.645 | 0.575 | 60.00% | 100.00% | 6 of 10 | 10 of 10 |
| 8 | ha1p_09663 | Gain | 0.5 | 0.3475 | 60.00% | 100.00% | 6 of 10 | 10 of 10 |
| 9 | ha1p_100558 | Gain | 0.665 | 0.495 | 77.78% | 77.78% | 7 of 9 | 7 of 9 |
| 10 | ha1p_10286 | Gain | 0.655 | 0.7575 | 60.00% | 100.00% | 6 of 10 | 10 of 10 |
| 11 | ha1p_108198 | Gain | 4.265 | −0.0275 | 30.00% | 88.89% | 3 of 10 | 8 of 9 |
| 12 | ha1p_16916 | Loss | 4.73 | −2.32 | 90.00% | 100.00% | 9 of 10 | 10 of 10 |
| 13 | ha1p_18823 | Gain | 0.615 | 1.02 | 88.89% | 88.89% | 8 of 9 | 8 of 9 |
| 14 | ha1p_22139 | Loss | 2 | −0.155 | 60.00% | 77.78% | 6 of 10 | 7 of 9 |
| 15 | ha1p_26420 | Gain | 2.105 | 0.925 | 77.78% | 88.89% | 7 of 9 | 8 of 9 |
| 16 | ha1p_38800 | Gain | 1.94 | 0.8525 | 100.00% | 70.00% | 10 of 10 | 7 of 10 |
| 17 | ha1p_41780 | Gain | 4.625 | 2.1975 | 80.00% | 100.00% | 8 of 10 | 10 of 10 |
| 18 | ha1p_42103 | Gain | 0.65 | 0.1275 | 10.00% | 100.00% | 1 of 10 | 10 of 10 |
| 19 | ha1p_47490 | Gain | 5.215 | 1.6825 | 50.00% | 100.00% | 5 of 10 | 10 of 10 |
| 20 | ha1p_47995 | Loss | 2.835 | −0.265 | 30.00% | 100.00% | 3 of 10 | 10 of 10 |
| 21 | ha1p_54181 | Gain | 0.55 | 0.5875 | 60.00% | 77.78% | 6 of 10 | 7 of 9 |
| 22 | ha1p_57326 | Gain | 2.195 | 0.7325 | 70.00% | 100.00% | 7 of 10 | 10 of 10 |
| 23 | ha1p_60271 | Gain | 1.52 | 0.685 | 80.00% | 66.67% | 4 of 5 | 2 of 3 |
| 24 | ha1p_62820 | Gain | 4.545 | 1.235 | 80.00% | 90.00% | 8 of 10 | 9 of 10 |
| 25 | ha1p_64271 | Gain | 0.635 | 0.0675 | 20.00% | 100.00% | 2 of 10 | 10 of 10 |
| 26 | ha1p_69412 | Gain | 3.18 | 1.6225 | 90.00% | 100.00% | 9 of 10 | 10 of 10 |
| 27 | ha1p_70432 | Gain | 3.22 | 0.3175 | 60.00% | 100.00% | 6 of 10 | 10 of 10 |
| 28 | ha1p_71854 | Gain | 5.48 | 1.42 | 90.00% | 100.00% | 9 of 10 | 10 of 10 |
| 29 | ha1p_81638 | Gain | 1.05 | 0.2 | 20.00% | 100.00% | 2 of 10 | 10 of 10 |
| 30 | ha1p_86556 | Gain | 2.105 | 0.88 | 88.89% | 83.33% | 8 of 9 | 5 of 6 |
| 31 | ha1p_91110 | Loss | 6 | −0.83 | 60.00% | 100.00% | 6 of 10 | 10 of 10 |
| 32 | ha1p_94558 | Gain | 0.71 | 0.57 | 90.00% | 70.00% | 9 of 10 | 7 of 10 |
| 33 | ha1p_96544 | Gain | 5.795 | 0.9375 | 80.00% | 90.00% | 8 of 10 | 9 of 10 |
| 34 | ha1p_97458 | Gain | 1.145 | 0.44 | 80.00% | 60.00% | 8 of 10 | 6 of 10 |
| 35 | ha1p_97786 | Gain | 0.82 | 1.045 | 85.71% | 100.00% | 6 of 7 | 2 of 2 |
| 36 | ha1p_98401 | Gain | 0.79 | 0.7625 | 70.00% | 100.00% | 7 of 10 | 10 of 10 |

TABLE 9
Sensitivity and specificity of loci identified as differentially methylated in breast tumors
among a panel of 10 ovarian tumor samples and 10 histologically normal ovary samples.

| | | | | Difference | | | | |
|---|---|---|---|---|---|---|---|---|
| Locus | | | | in Medians | | | Sensitivity | Specificity |
| Number | Feature Name | Type | Threshold | (T − N) | Sensitivity | Specificity | (n of n) | (n of n) |
| 1 | ha1c_00037 | Gain | 2.47 | 0.615 | 70.00% | 70.00% | 7 of 10 | 7 of 10 |
| 2 | ha1g_01283 | Gain | 1.13 | 0.1575 | 30.00% | 90.00% | 3 of 10 | 9 of 10 |
| 3 | ha1g_01465 | Loss | 0.795 | −0.0625 | 90.00% | 20.00% | 9 of 10 | 2 of 10 |
| 4 | ha1g_02335 | Gain | 5.265 | 1.405 | 60.00% | 90.00% | 6 of 10 | 9 of 10 |
| 5 | ha1g_04114 | Loss | 0.74 | −0.465 | 80.00% | 50.00% | 8 of 10 | 5 of 10 |
| 6 | ha1g_04194 | Gain | 0.865 | 2.385 | 90.00% | 80.00% | 9 of 10 | 8 of 10 |
| 7 | ha1p_05922 | Gain | 1.88 | 1.9475 | 80.00% | 90.00% | 8 of 10 | 9 of 10 |
| 8 | ha1p_09663 | Gain | 1.6 | 0.0375 | 20.00% | 90.00% | 2 of 10 | 9 of 10 |
| 9 | ha1p_100558 | Gain | 0.515 | 0.115 | 20.00% | 100.00% | 2 of 10 | 9 of 9 |
| 10 | ha1p_10286 | Gain | 0.93 | 0.075 | 70.00% | 50.00% | 7 of 10 | 5 of 10 |
| 11 | ha1p_108198 | Loss | 1.12 | −0.365 | 33.33% | 100.00% | 3 of 9 | 10 of 10 |
| 12 | ha1p_16916 | Loss | 4.005 | −3.5475 | 88.89% | 90.00% | 8 of 9 | 9 of 10 |
| 13 | ha1p_18823 | Gain | 2.175 | 0.145 | 11.11% | 100.00% | 1 of 9 | 9 of 9 |
| 14 | ha1p_22139 | Gain | 0.595 | 0.55 | 77.78% | 55.56% | 7 of 9 | 5 of 9 |
| 15 | ha1p_26420 | Gain | 1.49 | 1.8675 | 70.00% | 87.50% | 7 of 10 | 7 of 8 |
| 16 | ha1p_38800 | Gain | 2.68 | 0.7075 | 50.00% | 90.00% | 5 of 10 | 9 of 10 |
| 17 | ha1p_41780 | Gain | 4.41 | 0.815 | 77.78% | 60.00% | 7 of 9 | 6 of 10 |
| 18 | ha1p_42103 | Loss | 0.79 | −0.1325 | 100.00% | 20.00% | 10 of 10 | 2 of 10 |
| 19 | ha1p_47490 | Gain | 4.815 | −0.3125 | 40.00% | 100.00% | 4 of 10 | 10 of 10 |
| 20 | ha1p_47995 | Loss | 4.1 | −0.9275 | 70.00% | 80.00% | 7 of 10 | 8 of 10 |
| 21 | ha1p_54181 | Gain | 1.385 | 0 | 12.50% | 100.00% | 1 of 8 | 9 of 9 |
| 22 | ha1p_57326 | Gain | 1.485 | 0.38 | 66.67% | 80.00% | 6 of 9 | 8 of 10 |
| 23 | ha1p_60271 | Gain | 0.635 | 0.635 | 60.00% | 100.00% | 3 of 5 | 1 of 1 |
| 24 | ha1p_62820 | Gain | 5.055 | −0.0275 | 40.00% | 100.00% | 4 of 10 | 10 of 10 |
| 25 | ha1p_64271 | Loss | 0.8 | −0.035 | 90.00% | 10.00% | 9 of 10 | 1 of 10 |
| 26 | ha1p_69412 | Loss | 4.18 | −0.4425 | 50.00% | 100.00% | 5 of 10 | 10 of 10 |
| 27 | ha1p_70432 | Loss | 3.465 | −0.24 | 60.00% | 90.00% | 6 of 10 | 9 of 10 |
| 28 | ha1p_71854 | Gain | 2.915 | 2.82 | 66.67% | 90.00% | 6 of 9 | 9 of 10 |
| 29 | ha1p_81638 | Gain | 0.505 | 0.1525 | 40.00% | 90.00% | 4 of 10 | 9 of 10 |
| 30 | ha1p_86556 | Gain | 1.7 | 0.5475 | 50.00% | 83.33% | 4 of 8 | 5 of 6 |
| 31 | ha1p_91110 | Loss | 6 | −2.2825 | 80.00% | 87.50% | 8 of 10 | 7 of 8 |
| 32 | ha1p_94558 | Gain | 0.82 | 0.2025 | 50.00% | 80.00% | 5 of 10 | 8 of 10 |
| 33 | ha1p_96544 | Loss | 5.36 | −0.1 | 30.00% | 90.00% | 3 of 10 | 9 of 10 |
| 34 | ha1p_97458 | Loss | 1.475 | −0.5075 | 70.00% | 70.00% | 7 of 10 | 7 of 10 |
| 35 | ha1p_97786 | Gain | 0.52 | 0.085 | 28.57% | 100.00% | 2 of 7 | 4 of 4 |
| 36 | ha1p_98401 | Gain | 2.13 | 0.025 | 30.00% | 90.00% | 3 of 10 | 9 of 10 |

### Example 6

### Analysis of Loci Discovered to be Differentially DNA methylated in lung Cancer Among Breast and Ovarian Tumor and Histologically Normal Samples

The differential DNA methylation status of 36 loci found to be differentially DNA methylated in lung tumors relative to histologically normal lung samples (Table 6) was monitored in a randomly selected panel of 10 breast tumor samples and 10 histologically normal breast samples (Table 10). The same loci were analyzed in a randomly selected panel of 10 ovarian tumor samples and 10 histologically normal ovary samples (Table 11). Each sample was split into two equal portions of 3 μg. One portion was digested with McrBC (Treated Portion) in a total volume of 150 μL including 1×NEB2 buffer (New England Biolabs), 0.1 mg/mL bovine serum albumin (New England Biolabs), 2 mM GTP (Roche) and 48 units McrBC (New England Biolabs). The second portion was mock treated under identical conditions, except that 4.8 μL sterile 50% glycerol was added instead of McrBC enzyme (Untreated Portion). Samples were incubated at 37° C. for approximately 12 hours, followed by incubation at 60° C. to inactivate the McrBC enzyme. qPCR reactions and data analysis were performed as described in Example 2.

Sensitivity and specificity were calculated using ROC analysis derived thresholds as described above. Table 10 indicates the percent sensitivity and specificity for each locus analyzed in the panel of breast tumor and histologically normal breast samples. Table 11 indicates the percent sensitivity and specificity for each locus analyzed in the panel of ovarian tumor and histologically normal ovary samples. The number of samples scoring as positive for the methylation change among the analyzed tumor samples is indicated (Sensitivity (n of n)). For example, “7 of 10” indicates that seven tumor samples scored in the positive range as determined by ROC based average dCt thresholds (Threshold) among a total of 10 tumor samples analyzed. The number of samples scoring as negative for the methylation change among the analyzed histologically normal samples is also indicated (Specificity (n of n)).

TABLE 10
Sensitivity and specificity of loci identified as differentially methylated in lung tumors
among a panel of 10 breast tumor samples and 10 histologically normal breast samples.

| Locus | | | | Difference in | | | Sensitivity | Specificity |
|---|---|---|---|---|---|---|---|---|
| Number | Feature Name | Type | Threshold | Medians (T − N) | Sensitivity | Specificity | (n of n) | (n of n) |
| 37 | ha1g_00353 | Gain | 1.065 | 0.44 | 30.00% | 100.00% | 3 of 10 | 10 of 10 |
| 38 | ha1p_00553 | Loss | 1.6 | −0.065 | 90.00% | 40.00% | 9 of 10 | 4 of 10 |
| 39 | ha1p_04444 | Loss | 1.15 | −0.445 | 80.00% | 60.00% | 8 of 10 | 6 of 10 |
| 40 | ha1p_07264 | Gain | 0.805 | 0.3275 | 50.00% | 70.00% | 5 of 10 | 7 of 10 |
| 41 | ha1p_08159 | Loss | 6 | −0.415 | 90.00% | 20.00% | 9 of 10 | 2 of 10 |
| 42 | ha1p_103437 | Gain | 3.925 | 0.4375 | 100.00% | 40.00% | 10 of 10 | 4 of 10 |
| 43 | ha1p_105187 | Gain | 1.525 | 0.885 | 60.00% | 90.00% | 6 of 10 | 9 of 10 |
| 44 | ha1p_105778 | Loss | 1.745 | −1.6175 | 50.00% | 90.00% | 5 of 10 | 9 of 10 |
| 45 | ha1p_10757 | Gain | 1.475 | 2.1475 | 100.00% | 80.00% | 10 of 10 | 8 of 10 |
| 46 | ha1p_108911 | Loss | 1.475 | −0.47 | 60.00% | 60.00% | 6 of 10 | 6 of 10 |
| 47 | ha1p_111312 | Loss | 2.875 | −0.365 | 90.00% | 50.00% | 9 of 10 | 5 of 10 |
| 48 | ha1p_12483 | Gain | 2.26 | 0.4225 | 90.00% | 50.00% | 9 of 10 | 5 of 10 |
| 49 | ha1p_16097 | Gain | 0.54 | 0.3375 | 40.00% | 90.00% | 4 of 10 | 9 of 10 |
| 50 | ha1p_27029 | Gain | 1.925 | 0.9975 | 60.00% | 90.00% | 6 of 10 | 9 of 10 |
| 51 | ha1p_29823 | Gain | 0.685 | 0.485 | 60.00% | 70.00% | 6 of 10 | 7 of 10 |
| 52 | ha1p_40588 | Gain | 0.62 | 0.3325 | 50.00% | 70.00% | 5 of 10 | 7 of 10 |
| 53 | ha1p_45692 | Loss | 2.64 | −0.48 | 20.00% | 100.00% | 2 of 10 | 10 of 10 |
| 54 | ha1p_47429 | Gain | 1.48 | 0.97 | 70.00% | 90.00% | 7 of 10 | 9 of 10 |
| 55 | ha1p_49581 | Loss | 1.665 | −0.3725 | 80.00% | 50.00% | 8 of 10 | 5 of 10 |
| 56 | ha1p_55371 | Gain | 0.94 | 0.4025 | 90.00% | 50.00% | 9 of 10 | 5 of 10 |
| 57 | ha1p_58788 | Loss | 0.8 | −1.13 | 60.00% | 100.00% | 3 of 5 | 10 of 10 |
| 58 | ha1p_59216 | Gain | 2.14 | 1.16 | 66.67% | 88.89% | 6 of 9 | 8 of 9 |
| 59 | ha1p_61568 | Gain | 1.595 | 1.1975 | 66.67% | 100.00% | 4 of 6 | 6 of 6 |
| 60 | ha1p_61745 | Gain | 2.355 | 0.605 | 80.00% | 80.00% | 8 of 10 | 8 of 10 |
| 61 | ha1p_62060 | Gain | 1.335 | 1.145 | 80.00% | 60.00% | 8 of 10 | 6 of 10 |
| 62 | ha1p_62154 | Gain | 2.015 | −0.055 | 20.00% | 100.00% | 2 of 10 | 10 of 10 |
| 63 | ha1p_62869 | Gain | 1.265 | 0.7 | 90.00% | 60.00% | 9 of 10 | 6 of 10 |
| 64 | ha1p_64529 | Gain | 6 | 0 | 100.00% | 33.33% | 9 of 9 | 3 of 9 |
| 65 | ha1p_77581 | Gain | 0.615 | 0.9425 | 100.00% | 70.00% | 10 of 10 | 7 of 10 |
| 66 | ha1p_78965 | Gain | 1.065 | 0.065 | 60.00% | 60.00% | 6 of 10 | 6 of 10 |
| 67 | ha1p_80400 | Gain | 6 | 0 | 100.00% | 28.57% | 8 of 8 | 2 of 7 |
| 68 | ha1p_81949 | Gain | 0.715 | 0.59 | 100.00% | 70.00% | 10 of 10 | 7 of 10 |
| 69 | ha1p_82549 | Gain | 0.93 | 1.2775 | 100.00% | 60.00% | 10 of 10 | 6 of 10 |
| 70 | ha1p_84580 | Loss | 1.115 | −0.245 | 42.86% | 90.00% | 3 of 7 | 9 of 10 |
| 71 | ha1p_86042 | Gain | 0.63 | 0.1525 | 100.00% | 40.00% | 10 of 10 | 4 of 10 |
| 72 | ha1p_95305 | Gain | 3.26 | 0.8175 | 70.00% | 60.00% | 7 of 10 | 6 of 10 |

TABLE 11
Sensitivity and specificity of loci identified as differentially methylated in lung tumors
among a panel of 10 ovarian tumor samples and 10 histologically normal ovary samples.

| Locus | | | | Difference in | | | | |
|---|---|---|---|---|---|---|---|---|
| Number | Feature Name | Type | Threshold | Medians (T − N) | Sensitivity | Specificity | Sensitivity | Specificity |
| 37 | ha1g_00353 | Gain | 0.56 | 1.295 | 66.67% | 100.00% | 6 of 9 | 10 of 10 |
| 38 | ha1p_00553 | Loss | 0.9 | −0.3775 | 90.00% | 40.00% | 9 of 10 | 4 of 10 |
| 39 | ha1p_04444 | Gain | 0.65 | 0.195 | 50.00% | 90.00% | 5 of 10 | 9 of 10 |
| 40 | ha1p_07264 | Gain | 0.65 | 0.3225 | 50.00% | 100.00% | 5 of 10 | 10 of 10 |
| 41 | ha1p_08159 | Loss | 5.495 | −2.2775 | 88.89% | 80.00% | 8 of 9 | 8 of 10 |
| 42 | ha1p_103437 | Loss | 2.535 | −0.9 | 40.00% | 100.00% | 4 of 10 | 10 of 10 |
| 43 | ha1p_105187 | Gain | 0.8 | 0.88 | 90.00% | 90.00% | 9 of 10 | 9 of 10 |
| 44 | ha1p_105778 | Gain | 1.525 | 0.655 | 50.00% | 100.00% | 5 of 10 | 10 of 10 |
| 45 | ha1p_10757 | Gain | 1.57 | 2.235 | 70.00% | 100.00% | 7 of 10 | 10 of 10 |
| 46 | ha1p_108911 | Gain | 1.675 | 0.1525 | 60.00% | 70.00% | 6 of 10 | 7 of 10 |
| 47 | ha1p_111312 | Gain | 1.885 | 0.7375 | 60.00% | 100.00% | 6 of 10 | 10 of 10 |
| 48 | ha1p_12483 | Gain | 4.095 | −0.215 | 40.00% | 80.00% | 4 of 10 | 8 of 10 |
| 49 | ha1p_16097 | Gain | 1.12 | 0.0575 | 30.00% | 100.00% | 3 of 10 | 10 of 10 |
| 50 | ha1p_27029 | Gain | 0.69 | 1.85 | 80.00% | 100.00% | 8 of 10 | 10 of 10 |
| 51 | ha1p_29823 | Gain | 0.85 | 0.48 | 50.00% | 100.00% | 5 of 10 | 10 of 10 |
| 52 | ha1p_40588 | Gain | 0.57 | 0.34 | 22.22% | 100.00% | 2 of 9 | 10 of 10 |
| 53 | ha1p_45692 | Loss | 5.855 | −0.71 | 60.00% | 100.00% | 6 of 10 | 10 of 10 |
| 54 | ha1p_47429 | Gain | 0.72 | 0.97 | 90.00% | 100.00% | 9 of 10 | 10 of 10 |
| 55 | ha1p_49581 | Loss | 0.795 | −0.2 | 30.00% | 90.00% | 3 of 10 | 9 of 10 |
| 56 | ha1p_55371 | Gain | 1 | 0.28 | 70.00% | 70.00% | 7 of 10 | 7 of 10 |
| 57 | ha1p_58788 | Gain | 1.18 | 1.7525 | 90.00% | 100.00% | 9 of 10 | 10 of 10 |
| 58 | ha1p_59216 | Gain | 1.93 | 1.3275 | 70.00% | 100.00% | 7 of 10 | 10 of 10 |
| 59 | ha1p_61568 | Gain | 2.875 | 3.595 | 83.33% | 80.00% | 5 of 6 | 4 of 5 |
| 60 | ha1p_61745 | Gain | 1.455 | 1.445 | 90.00% | 100.00% | 9 of 10 | 10 of 10 |
| 61 | ha1p_62060 | Gain | 5.235 | 1.47 | 44.44% | 100.00% | 4 of 9 | 10 of 10 |
| 62 | ha1p_62154 | Gain | 0.895 | 0.08 | 30.00% | 100.00% | 3 of 10 | 10 of 10 |
| 63 | ha1p_62869 | Gain | 0.535 | 1.49 | 80.00% | 100.00% | 8 of 10 | 9 of 9 |
| 64 | ha1p_64529 | Loss | 6 | 0 | 0.00% | 100.00% | 0 of 9 | 8 of 8 |
| 65 | ha1p_77581 | Gain | 1.235 | 0.18 | 44.44% | 100.00% | 4 of 9 | 10 of 10 |
| 66 | ha1p_78965 | Gain | 0.675 | 0.45 | 70.00% | 100.00% | 7 of 10 | 10 of 10 |
| 67 | ha1p_80400 | Loss | 6 | 0 | 14.29% | 100.00% | 1 of 7 | 8 of 8 |
| 68 | ha1p_81949 | Gain | 1.15 | 1.475 | 77.78% | 100.00% | 7 of 9 | 10 of 10 |
| 69 | ha1p_82549 | Gain | 2.385 | 0.895 | 66.67% | 100.00% | 6 of 9 | 10 of 10 |
| 70 | ha1p_84580 | Loss | 0.805 | −0.29 | 44.44% | 100.00% | 4 of 9 | 10 of 10 |
| 71 | ha1p_86042 | Loss | 1.05 | −0.165 | 33.33% | 100.00% | 3 of 9 | 10 of 10 |
| 72 | ha1p_95305 | Loss | 3.355 | 0.01 | 33.33% | 90.00% | 3 of 9 | 9 of 10 |

### Example 7

### Analysis of Loci Discovered to be Differentially DNA Methylated in Ovarian Cancer Among Breast and Lung Tumor and Histologically Normal Samples

The differential DNA methylation status of 24 loci found to be differentially DNA methylated in lung tumors relative to histologically normal lung samples (Table 7) was monitored in a randomly selected panel of 10 breast tumor samples and 10 histologically normal breast samples (Table 12). The same loci were analyzed in a randomly selected panel of 10 lung tumor samples and 10 histologically normal lung samples (Table 13). Each sample was split into two equal portions of 3 μg. One portion was digested with McrBC (Treated Portion) in a total volume of 150 μL including 1×NEB2 buffer (New England Biolabs), 0.1 mg/mL bovine serum albumin (New England Biolabs), 2 mM GTP (Roche) and 48 units McrBC (New England Biolabs). The second portion was mock treated under identical conditions, except that 4.8 μL sterile 50% glycerol was added instead of McrBC enzyme (Untreated Portion). Samples were incubated at 37° C. for approximately 12 hours, followed by incubation at 60° C. to inactivate the McrBC enzyme. qPCR reactions and data analysis were performed as described in Example 2.

Sensitivity and specificity were calculated using ROC analysis derived thresholds as described above. Table 12 indicates the percent sensitivity and specificity for each locus analyzed in the panel of breast tumor and histologically normal breast samples. Table 13 indicates the percent sensitivity and specificity for each locus analyzed in the panel of lung tumor and histologically normal lung samples. The number of samples scoring as positive for the methylation change among the analyzed tumor samples is indicated (Sensitivity (n of n)). For example, “7 of 10” indicates that seven tumor samples scored in the positive range as determined by ROC based average dCt thresholds (Threshold) among a total of 10 tumor samples analyzed. The number of samples scoring as negative for the methylation change among the analyzed histologically normal samples is also indicated (Specificity (n of n)).

As demonstrated in Tables 8-13, although a differential DNA methylation biomarker may have been initially discovered in an analysis of a particular cancer type, that biomarker has applications outside that specific cancer type. For example, the locus represented by Locus number 1 (hal c_{—}00037; DNA sequence region SEQ ID NO:385) was originally discovered in a microarray-based comparison of breast tumor and adjacent histologically normal breast tissue, and this differentially methylated locus was subsequently found to display approximately 70% sensitivity and 96% specificity for discriminating between breast tumor and normal breast tissue. However, the same locus also displayed 70% sensitivity and 100% specificity for discriminating between lung tumor and histologically normal lung tissue and 70% sensitivity and 70% specificity for discriminating between ovarian tumor and histologically normal ovary tissue. Therefore, DNA methylation based biomarkers initially identified in an analysis of a particular cancer type can be useful in the detection or diagnosis of additional cancer types.

TABLE 12
Sensitivity and specificity of loci identified as differentially methylated in ovarian tumors
among a panel of 10 breast tumor samples and 10 histologically normal breast samples.

| | | | | Difference | | | | |
|---|---|---|---|---|---|---|---|---|
| Locus | | | | in Medians | | | Sensitivity | Specificity |
| Number | Feature Name | Type | Threshold | (T − N) | Sensitivity | Specificity | (n of n) | (n of n) |
| 73 | CHR01P152508183 | Gain | 1.77 | 2.0225 | 100.00% | 70.00% | 10 of 10 | 7 of 10 |
| 74 | CHR02P046721735 | Gain | 5.06 | 3.09 | 90.00% | 90.00% | 9 of 10 | 9 of 10 |
| 75 | CHR04P001292657 | Gain | 6 | 0.32 | 100.00% | 55.56% | 9 of 9 | 5 of 9 |
| 76 | CHR05P043085585 | Loss | 1.11 | −0.24 | 100.00% | 40.00% | 10 of 10 | 4 of 10 |
| 77 | CHR08P097127672 | Gain | 0.61 | 0.84 | 80.00% | 70.00% | 8 of 10 | 7 of 10 |
| 78 | CHR08P102461728 | Loss | 2.82 | −0.0825 | 100.00% | 30.00% | 10 of 10 | 3 of 10 |
| 79 | CHR08P143804195 | Gain | 6 | 0 | 100.00% | 20.00% | 10 of 10 | 2 of 10 |
| 80 | CHR09P021979668 | Gain | 3.725 | 1.02 | 90.00% | 80.00% | 9 of 10 | 8 of 10 |
| 81 | CHR09P067743642 | Gain | 1.595 | 1.4075 | 100.00% | 62.50% | 9 of 9 | 5 of 8 |
| 82 | CHR11P010436241 | Gain | 2.59 | 1.595 | 80.00% | 70.00% | 8 of 10 | 7 of 10 |
| 83 | CHR11P117233022 | Gain | 2.95 | 1.075 | 100.00% | 70.00% | 10 of 10 | 7 of 10 |
| 84 | CHR12P044081945 | Gain | 3.135 | 2.015 | 80.00% | 90.00% | 8 of 10 | 9 of 10 |
| 85 | CHR13P042532794 | Gain | 2.775 | 1.5675 | 100.00% | 70.00% | 10 of 10 | 7 of 10 |
| 86 | CHR14P049549993 | Gain | 2.545 | 1.9625 | 100.00% | 60.00% | 10 of 10 | 6 of 10 |
| 87 | CHR15P062682028 | Gain | 6 | 0 | 100.00% | 40.00% | 7 of 7 | 2 of 5 |
| 88 | CHR16P070471895 | Gain | 1.9 | 0.66 | 85.71% | 60.00% | 6 of 7 | 6 of 10 |
| 89 | CHR17P007309455 | Loss | 1.795 | 0.235 | 100.00% | 33.33% | 3 of 3 | 1 of 3 |
| 90 | CHR19P047620296 | Gain | 5.77 | 1.155 | 100.00% | 70.00% | 10 of 10 | 7 of 10 |
| 91 | CHR19P054350430 | Gain | 3.29 | 1.61 | 90.00% | 70.00% | 9 of 10 | 7 of 10 |
| 92 | CHR19P059796623 | Loss | 4.205 | −0.9025 | 80.00% | 70.00% | 8 of 10 | 7 of 10 |
| 93 | CHR20P038041321 | Gain | 1.13 | 1.56 | 100.00% | 80.00% | 10 of 10 | 8 of 10 |
| 94 | ha1p_108204_l50 | Gain | 0.53 | −0.0925 | 77.78% | 37.50% | 7 of 9 | 3 of 8 |
| 95 | ha1p_48631_l50 | Loss | 1.075 | 1.1925 | 30.00% | 100.00% | 3 of 10 | 10 of 10 |
| 96 | ha1p_94692_l50 | Gain | 3.02 | 0.7575 | 50.00% | 90.00% | 4 of 8 | 9 of 10 |

Although the invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

All publications, databases, Genbank sequences, patents, and patent applications cited in this specification are herein incorporated by reference as if each was specifically and individually indicated to be incorporated by reference.

TABLE 13
Sensitivity and specificity of loci identified as differentially methylated in ovarian
tumors among a panel of 10 lung tumor samples and 10 histologically normal lung samples.

| Locus | | | | Difference in | | | Sensitivity | Specificity |
|---|---|---|---|---|---|---|---|---|
| Number | Feature Name | Type | Threshold | Medians (T − N) | Sensitivity | Specificity | (n of n) | (n of n) |
| 73 | CHR01P152508183 | Gain | 2.75 | 1.5475 | 80.00% | 90.00% | 8 of 10 | 9 of 10 |
| 74 | CHR02P046721735 | Gain | 2.98 | 0.56 | 70.00% | 90.00% | 7 of 10 | 9 of 10 |
| 75 | CHR04P001292657 | Gain | 6 | 0.4275 | 100.00% | 70.00% | 10 of 10 | 7 of 10 |
| 76 | CHR05P043085585 | Loss | 0.915 | 0.02 | 90.00% | 10.00% | 9 of 10 | 1 of 10 |
| 77 | CHR08P097127672 | Gain | 1.59 | 0.07 | 20.00% | 100.00% | 2 of 10 | 10 of 10 |
| 78 | CHR08P102461728 | Loss | 1.775 | −0.38 | 90.00% | 60.00% | 9 of 10 | 6 of 10 |
| 79 | CHR08P143804195 | Gain | 6 | 0 | 100.00% | 0.00% | 10 of 10 | 0 of 10 |
| 80 | CHR09P021979668 | Gain | 1.885 | 0.31 | 80.00% | 80.00% | 8 of 10 | 8 of 10 |
| 81 | CHR09P067743642 | Gain | 2.615 | 0.23 | 77.78% | 80.00% | 7 of 9 | 8 of 10 |
| 82 | CHR11P010436241 | Loss | 1.215 | −0.3875 | 40.00% | 100.00% | 4 of 10 | 10 of 10 |
| 83 | CHR11P117233022 | Gain | 2.65 | 0.0525 | 30.00% | 100.00% | 3 of 10 | 10 of 10 |
| 84 | CHR12P044081945 | Gain | 3.3 | 1.055 | 80.00% | 90.00% | 8 of 10 | 9 of 10 |
| 85 | CHR13P042532794 | Gain | 3.27 | 0.11 | 30.00% | 100.00% | 3 of 10 | 10 of 10 |
| 86 | CHR14P049549993 | Gain | 3.515 | 0.0275 | 30.00% | 100.00% | 3 of 10 | 10 of 10 |
| 87 | CHR15P062682028 | Loss | 5.9 | 0 | 12.50% | 100.00% | 1 of 8 | 7 of 7 |
| 88 | CHR16P070471895 | Loss | 1.89 | −0.43 | 70.00% | 77.78% | 7 of 10 | 7 of 9 |
| 89 | CHR17P007309455 | Loss | 3.245 | −2.3925 | 66.67% | 100.00% | 2 of 3 | 2 of 2 |
| 90 | CHR19P047620296 | Gain | 5.615 | 0.8275 | 90.00% | 90.00% | 9 of 10 | 9 of 10 |
| 91 | CHR19P054350430 | Gain | 3.48 | −0.035 | 40.00% | 80.00% | 4 of 10 | 8 of 10 |
| 92 | CHR19P059796623 | Loss | 3.505 | −1.0175 | 80.00% | 80.00% | 8 of 10 | 8 of 10 |
| 93 | CHR20P038041321 | Gain | 0.605 | 0.4175 | 80.00% | 70.00% | 8 of 10 | 7 of 10 |
| 94 | ha1p_108204_l50 | Loss | 1.54 | −0.2825 | 80.00% | 50.00% | 8 of 10 | 5 of 10 |
| 95 | ha1p_48631_l50 | Loss | 4.035 | −1.3975 | 60.00% | 90.00% | 6 of 10 | 9 of 10 |
| 96 | ha1p_94692_l50 | Gain | 3.15 | 0.115 | 55.56% | 66.67% | 5 of 9 | 6 of 9 |

What is claimed is:

1. A method for determining the methylation status of an individual, the method comprising:
obtaining a biological sample from an individual; and
determining the methylation status of at least one cytosine within a DNA region in a sample from the individual where the DNA region is at least 90% identical to a sequence selected from the group consisting of SEQ ID NO: 391, 385, 386, 387, 388, 389, 390, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, and 480.

2. The method of claim 1, wherein the determining step comprises determining the methylation status of at least one cytosine in the DNA region corresponding to a nucleotide in a biomarker, wherein the biomarker is at least 90% identical to a sequence selected from the group consisting of SEQ ID NO: 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, and 384.

3. The method of claim 2, wherein the determining step comprises determining the methylation status of the DNA region corresponding to the biomarker.

4. The method of claim 1, wherein the sample is from blood serum, blood plasma, urine, sputum, or tissue biopsy.

5. The method of claim 1, wherein the methylation status of at least one cytosine is compared to the methylation status of a control locus.

6. The method of claim 5, wherein the control locus is an endogenous control.

7. The method of claim 5, wherein the control locus is an exogenous control.

8. The method of claim 1, wherein the determining step comprises determining the methylation status of at least one cytosine in at least two DNA regions.

9. A method for determining the presence or absence of cancer in an individual, the method comprising:
a) determining the methylation status of at least one cytosine within a DNA region in a sample from the individual where the DNA region is at least 90% identical to a sequence selected from the group consisting of SEQ ID NO: 391, 385, 386, 387, 388, 389, 390, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, and 480;
b) comparing the methylation status of the at least one cytosine to a threshold value for the at least one cytosine, wherein the threshold value distinguishes between individuals with and without cancer, wherein the comparison of the methylation status to the threshold value is predictive of the presence or absence of cancer in the individual.

10. The method of claim 9, wherein the determining step comprises determining the methylation status of at least one cytosine in the DNA region corresponding to a nucleotide in a biomarker, wherein the biomarker is at least 90% identical to a sequence selected from the group consisting of SEQ ID NO: 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, and 384.

11. The method of claim 10, wherein the determining step comprises determining the methylation status of the DNA region corresponding to the biomarker.

12. The method of claim 9, wherein the sample is from blood serum, blood plasma, urine, sputum, or a tissue biopsy.

13. The method of claim 9, wherein the methylation status of at least one biomarker from the list is compared to the methylation value of a control locus.

14. The method of claim 13, wherein the control locus is an endogenous control.

15. The method of claim 13, wherein the control locus is an exogenous control.

16. The method of claim 9, wherein the determining step comprises determining the methylation status of at least one cytosine from at least two DNA regions.

17. A computer implemented method for determining the presence or absence of cancer in an individual, the method comprising:
receiving, at a host computer, a methylation value representing the methylation status of at least one cytosine within a DNA region in a sample from the individual where the DNA region is at least 90% identical to a sequence selected from the group consisting of SEQ ID NO: 391, 385, 386, 387, 388, 389, 390, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, and 480; and
comparing, in the host computer, the methylation value to a threshold value, wherein the threshold value distinguishes between individuals with and without cancer, wherein the comparison of the methylation value to the threshold value is predictive of the presence or absence of cancer in the individual.

18. The method of claim 17, wherein the receiving step comprises receiving at least two methylation values, the two methylation values representing the methylation status of at least one cytosine biomarker from two different DNA regions; and
the comparing step comprises comparing the methylation values to one or more threshold value(s) wherein the threshold value distinguishes between individuals with and without cancer, wherein the comparison of the methylation value to the threshold value is predictive of the presence or absence of cancer in the individual.

19. A computer program product for determining the presence or absence of cancer in an individual, the computer readable product comprising:
a computer readable medium encoded with program code, the program code including:
program code for receiving a methylation value representing the methylation status of at least one cytosine within a DNA region in a sample from the individual where the DNA region is at least 90% identical to a sequence selected from the group consisting of SEQ ID NO: 391, 385, 386, 387, 388, 389, 390, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, and 480; and
program code for comparing the methylation value to a threshold value, wherein the threshold value distinguishes between individuals with and without cancer, wherein the comparison of the methylation value to the threshold value is predictive of the presence or absence of cancer in the individual.

20. A kit for determining the methylation status of at least one biomarker, the kit comprising:
(1) a pair of polynucleotides capable of specifically amplifying at least a portion of a DNA region where the DNA region is at least 90% identical to a sequence selected from the group consisting of SEQ ID NO: 391, 385, 386, 387, 388, 389, 390, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, and 480; and
a methylation-dependent or methylation sensitive restriction enzyme and/or sodium bisulfite; or
(2) sodium bisulfite, primers and adapters for whole genome amplification, and polynucleotides to quantify the presence of the converted methylated and/or the converted unmethylated sequence of at least one cytosine from a DNA region that is at least 90% identical to a sequence selected from the group consisting of SEQ ID NO: 391, 385, 386, 387, 388, 389, 390, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, and 480; or
(3) methylation sensing restriction enzymes, primers and adapters for whole genome amplification, and polynucleotides to quantify the number of copies of at least a portion of a DNA region where the DNA region is at least 90% identical to a sequence selected from the group consisting of SEQ ID NO: 391, 385, 386, 387, 388, 389, 390, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, and 480; or
(4) a methylation sensing binding moiety and polynucleotides to quantify the number of copies of at least a portion of a DNA region where the DNA region is at least 90% identical to a sequence selected from the group consisting of SEQ ID NO: 391, 385, 386, 387, 388, 389, 390, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, and 480.

21. The kit of claim 20, wherein the pair of polynucleotides are capable of specifically amplifying a biomarker that is at least 90% identical to a sequence selected from the group consisting of SEQ ID NOs: 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, and 384.

22. The kit of claim 20, wherein the kit comprises at least two pairs of polynucleotides, wherein each pair is capable of specifically amplifying at least a portion of a different DNA region.

23. The kit of claim 20, wherein the kit further comprises a detectably labeled polynucleotide probe that specifically detects the amplified biomarker in a real time amplification reaction.
